(19) United States
(12) Patent Application Publication
(10) Pub. No.: US 2022/0256822 A1
(43) Pub. Date: Aug. 18, 2022

(54) GENETIC MODIFICATION NON-HUMAN ORGANISM, EGG CELLS, FERTILIZED EGGS, AND METHOD FOR MODIFYING TARGET GENES

(71) Applicant: JAPAN SCIENCE AND TECHNOLOGY AGENCY, Kawaguchi-shi (JP)

(72) Inventors: Takayuki SHINDO, Matsumoto-shi (JP); Takayuki SAKURAI, Matsumoto-shi (JP)

(73) Assignee: JAPAN SCIENCE AND TECHNOLOGY AGENCY, Kawaguchi-shi (JP)

(21) Appl. No.: 17/661,097

(22) Filed: Apr. 28, 2022

Related U.S. Application Data
(62) Division of application No. 16/061,769, filed on Jun. 13, 2018, now abandoned.

(30) Foreign Application Priority Data
Dec. 18, 2015 (JP) 2015-247960

(51) Int. Cl. A01K 67/027; C12N 9/22; C12N 15/90; C12N 15/873

(52) U.S. Cl. CPC A01K 67/0278; C12N 9/22; A01K 67/027; C12N 15/907; A01K 67/0276; A01K 2267/0375; A01K 2267/01; C12N 2310/20; A01K 2227/105; A01K 2227/107; A01K 2267/0331; C12N 15/873

(57) ABSTRACT

The present invention provides a genetic modification non-human organism in which an expression level of Cas9 is high and a plurality of different genes or a plurality of different locations in the same gene can be edited at the same time with high efficiency. The genetic modification non-human organism of the present invention includes a nuclear genome having at least 3 copies of genes that code for Cas9 (CRISPR-associated 9). Egg cells of the present invention are derived from the genetic modification non-human organism having the nuclear genome into which at least 3 copies of genes that code for the Cas9 are introduced. Fertilized eggs of the present invention are obtained by fertilizing the egg cells and sperm derived from the same species of the organism. A method for modifying target genes of the present invention includes a step of introducing a guide RNA into cells derived from the genetic modification non-human organism, the egg cells, or the fertilized eggs.

### CROSS REFERENCE TO RELATED APPLICATIONS

This application is a divisional of U.S. application Ser. No. 16/061,769, filed on Jun. 13, 2018, which is a national stage patent application of international patent application PCT/JP2016/085391, filed on Nov. 29, 2016, the text of which is incorporated by reference, and claims foreign priority to Japanese Patent Application No. 2015-247960, filed on Dec. 18, 2015, the entire content of which is incorporated herein by reference.

### TECHNICAL FIELD

The present invention relates to a genetic modification non-human organism, egg cells, fertilized eggs, and a method for modifying target genes.

Priority is claimed on Japanese Patent Application No. 2015-247960, filed on Dec. 18, 2015, the content of which is incorporated herein by reference.

### BACKGROUND ART

In research on disease mechanisms and development research for a new drug, the existence of an animal disease model bearing pathologies of the disease holds the key to the success of the research. In regard to genetic factors of a human disease, it has become possible to relatively easily obtain genetic information for several million locations and an SNP locus with genome analysis techniques undergoing rapid development in recent years. Many loci of disease-associated genes have been identified and reported for many diseases using the technique. On the other hand, even in a case where genetic modification mice having such disease candidate genes are produced, a phenotype is not exhibited in most cases. This is because an abnormality in a single gene does not necessarily explain pathologies in which multiple factors are involved, such as lifestyle diseases. Accordingly, it is considered that an effect of drug development targeting each of these candidate genes is limited. In order to solve such problems, there is a need to prepare a system to quickly and systematically prepare and utilize an animal disease model of multilocus modification, but in a method for genetic modification by ES cells of the related art, only genetic modification of a single operation, one locus, and a gene at one location was possible, and therefore it was difficult to realize the above solution.

Recently, a genetic modification technique by CRISPR (Clustered Regularly Interspaced Short Palindromic Repeats) system has appeared. The CRISPR system has the potential of a novelty that transcends the ES cell-mediated genetic modification technique of the related art. In the CRISPR system, three types of RNAs or DNAs, which are a guide RNA that guides cleavage of a target gene, an mRNA of Cas9 (CRISPR-associated 9) which is a nuclease actually involved in the cleavage, and a modified gene that is homologously inserted into a gene region to be cleaved, are microinjected into a fertilized egg so as to develop the embryo thereof up to the adult cell, and therefore genetic modification mice can be obtained.

It is disclosed in NPL 1 that by using the CRISPR system, knockin of genes that code for Cas9 into a ROSA26 gene region of ES cells is performed, and thus Cas9 knockin mice can be produced.

### CITATION LIST

### Patent Literature

[NPL 1]: Randall J. Platt, et al., Cell, vol. 159, p. 440-455, 2014

### SUMMARY OF INVENTION

### Technical Problem

In the method for producing genetic modification mice utilizing the CRISPR system of the related art, the number of mRNA of Cas9 was large, and therefore amounts of other guide RNAs to be microinjected and modified genes were limited.

In addition, in the Cas9 knockin mice disclosed in NPL 1, because only one copy of the gene of Cas9 has been introduced, it was difficult to edit a plurality of different genes or a plurality of different locations in the same gene at the same time.

The present invention has been made in view of the above circumstances, and an object thereof is to provide a genetic modification non-human organism in which an expression level of Cas9 is high and a plurality of different genes or a plurality of different locations in the same gene can be edited at the same time with high efficiency. The present invention further provides a genetic modification technique which is simply and rapidly and site-specifically performed with respect to a target gene using the genetic modification non-human organism.

### Solution to Problem

That is, the present invention includes the following aspects.

[1] A genetic modification non-human organism, including a nuclear genome that has at least 3 copies of genes that code for Cas9 (CRISPR-associated 9).

[2] The genetic modification non-human organism according to [1], in which the Cas9 is a variant.

[3] The genetic modification non-human organism according to [2], in which the Cas9 is a variant lacking activity of cleaving one or both strands of a target gene containing a target sequence.

[4] The genetic modification non-human organism according to any one of [1] to [3], in which the nuclear genome includes a CAG promoter, an expression-inducible promoter, a viral promoter, or a tissue-specific promoter, which is operably linked to the genes that code for the Cas9.

[5] The genetic modification non-human organism according to any one of [1] to [4], in which the non-human organism is a mammal.

[6] The genetic modification non-human organism according to any one of [1] to [5], in which the genes that code for the Cas9 are tandemly introduced into the nuclear genome.

[7] Egg cells derived from a genetic modification non-human organism having a nuclear genome into which at least 3 copies of genes that code for Cas9 are introduced.

[8] The egg cells according to [7], in which in the nucleus of the egg cell, the nuclear genome into which the genes that code for the Cas9 are introduced is not contained.

[9] Fertilized eggs obtained by fertilizing the egg cells according to [7] or [8] and sperm derived from the same species of the organism.

[10] The fertilized eggs according to [9], in which in the nucleus of the egg cell, the nuclear genome into which genes that code for Cas9 are introduced is not contained.

[11] The fertilized eggs according to [9] or [10], in which the sperm is derived from the same species of the organism of a wild-type.

[12] A method for modifying target genes, including a step of introducing a guide RNA into cells derived from the genetic modification non-human organism according to any one of [1] to [6], the egg cells according to [7] or [8], or the fertilized eggs according to any one of [9] to [11].

[13] A method for modifying target genes, including:

a step of introducing a guide RNA into cells derived from the genetic modification non-human organism according to any one of [1] to [6], the egg cells according to [7] or [8], or the fertilized eggs according to any one of [9] to [11];

a step of cleaving the target genes at a cleavage site located upstream of a PAM (proto-spacer adjacent motif) sequence by Cas9 expressed in the cells derived from the genetic modification non-human organism, the egg cells derived from the genetic modification non-human organism, or the fertilized eggs derived from the genetic modification non-human organism; and

a step of obtaining the modified target genes in a region determined by complementary binding of the guide RNA and the target genes,

in which the target genes have the PAM sequence, and

the guide RNA contains a polynucleotide including a base sequence complementary to an upstream base sequence of the PAM sequence in the target genes.

[14] A method for modifying target genes, including:

a step of introducing a guide RNA into cells derived from the genetic modification non-human organism according to any one of [1] to [6], the egg cells according to [7] or [8], or the fertilized eggs according to any one of [9] to [11];

a step of cleaving the target genes at a cleavage site located three bases upstream of a PAM sequence by Cas9 expressed in the cells derived from the genetic modification non-human organism, the egg cells derived from the genetic modification non-human organism, or the fertilized eggs derived from the genetic modification non-human organism; and

a step of obtaining the modified target genes in a region determined by complementary binding of the guide RNA and the target genes,

in which the target genes have the PAM sequence including NGG (N is any one base selected from the group consisting of adenine, cytosine, thymine, and guanine), and

the guide RNA contains a polynucleotide including base sequences complementary to base sequences from one base upstream to 20 bases to 24 bases upstream of the PAM sequence in the target genes.

[15] The method for modifying target genes according to any one of [12] to [14], in which in the introduction step, the guide RNA is introduced into a living genetic modification non-human organism in a case where an introduction target is the cell derived from the genetic modification non-human organism.

[16] The method for modifying target genes according to any one of [12] to [14], further including a step of implanting the fertilized eggs into a uterus or an oviduct of the same species of the non-human animal so as to develop the eggs in a case where an introduction target is the fertilized egg derived from the genetic modification non-human animal in the introduction step.

[17] The method for modifying target genes according to any one of [12] to [16], in which in the introduction step, two or more of the guide RNAs are introduced.

[18] The method for modifying target genes according to any one of [12] to [17], in which in the introduction step, a foreign gene is introduced together with the guide RNAs.

[19] The method for modifying target genes according to [18], in which the foreign gene does not have the PAM sequence.

### Advantageous Effects of Invention

According to the genetic modification non-human organism, the egg cells, and the fertilized eggs of the present invention, a plurality of different genes or a plurality of different locations in the same gene can be edited at the same time with high efficiency. In addition, according to the egg cells and the fertilized eggs of the present invention, the genetic modification non-human organism can be produced rapidly at a low cost. Furthermore, the genetic modification non-human organism, the egg cells, and the fertilized eggs of the present invention can be utilized in studies of lifestyle diseases such as cancer or heart disease in which abnormality of many genes is involved, or disease-related genes identified by genome-wide association study (GWAS).

### BRIEF DESCRIPTION OF DRAWINGS

FIG. 1A is a schematic diagram showing each structure of NFCas9 (Cas9 gene having a NLS and a Flag tag) and FCas9 (Cas9 having only the Flag tag) in Example 1.

FIG. 1B is a graph comparing the copy number of Cas9 mRNA in 50 ng RNA in each tissue of a mouse of NFCas9-2 line in Test Example 1.

FIG. 1C is a graph showing changes in body weight of male or female mice (4 weeks old to 10 weeks old) having different genotypes of NFCas9-2 line in Test Example 1.

FIG. 1D shows images of results in which each tissue section of mice (10 weeks old) having different genotypes of NFCas9-2 line is stained with hematoxylin and eosin in Test Example 1.

FIG. 1E is a graph showing results of detecting an amount of a physiological marker in serum of the mouse (10 weeks old) having different genotypes of NFCas9-2 line in Test Example 1.

FIG. 2A is a table showing the copy number of Cas9 genes introduced in 4 lines of NFCas9 mouse and 5 lines of FCas9 mouse constructed in Example 1, sex, a genotype in F1 generation, presence or absence of Cas9 transmission into F1 generation, and an expression level of Cas9.

FIG. 2B is a graph showing a calibration curve for calculating the copy number of Cas9 introduced in 4 lines of NFCas9 mouse and 5 lines of FCas9 mouse constructed in Example 1.

FIG. 2C shows images of results of performing a western blot analysis using Flag tag on expression of Cas9 protein NACas9 mouse and FCas9 mouse constructed in Example 1.

FIG. 3A is an image showing results in which PCR products of SpPCR are detected by 1% agarose gel electrophoresis using genomic DNA of NFCas9-2 line in Example 1.

FIG. 3B is a schematic diagram showing a location where NFCas9 of NFCas9-2 line in Example 1 is inserted.

FIG. 3C is an image showing results in which PCR products obtained by a site-specific primer for confirming the results in FIG. 3B in Example 1 are detected by 1% agarose gel electrophoresis.

FIG. 4A is a diagram showing genotypes in fertilized eggs obtained by artificial fertilization of various crossing pairs in Test Example 2.

FIG. 4B is a table showing the number of viable eggs and the number of indel mutations in the fertilized eggs obtained by artificial fertilization of various crossing pairs in Test Example 2.

FIG. 4C is a schematic diagram showing a sequence of a guide RNA used for modification of Ramp2 gene in Test Example 2. FIG. 4C discloses SEQ ID NO: 83.

FIG. 4D is an image showing results of T7 endonuclease I (T7EI) assay in Test Example 2.

FIG. 5A is a table showing the number of viable zygotes and the number of indel mutations in Test Example 3.

FIG. 5B shows images of results of T7EI assay of the blastocysts in Test Example 3.

FIG. 6A is a table showing the number of viable eggs and the number of indel mutated embryos after 13.5 days to 15.5 days from implantation in Test Example 3.

FIG. 6B shows images of forms of embryos of ten pups after 13.5 days from implantation in Test Example 3.

FIG. 6C shows images of results of T7EI assay on blastocysts of the embryos after 13.5 days from implantation in Test Example 3.

FIG. 6D is a diagram showing results of sequences of Ramp1 gene of the embryos of ten pups after 13.5 days from implantation in Test Example 3. FIG. 6D discloses SEQ ID NOS: 84, 84-85, 84, 86, 84, 87, 84, 88, 84, 89, 84, 84, 84, 90, 84, 84, 84, 84, 91, 91-92, 91, 93, 91, 94, 91, 95, 91, 96, 91, 91, 91, 97, 98, 99, 91, 91, 91, 100, and 91, respectively, in order of appearance.

FIG. 6E is a diagram showing relative RNA expression levels in Ramp1 gene and Ramp2 gene of the embryos of ten pups after 13.5 days from implantation in Test Example 3.

FIG. 7A shows a table and a graph showing determination results of genotypes and the presence or absence of mutations in Test Example 4.

FIG. 7B is an image showing results of T7EI assay of the blastocysts in Test Example 4.

FIG. 8 is a schematic diagram showing sequences of guide RNAs used for modification of genes in Test Example 5. FIG. 8 discloses SEQ ID NOS: 101-107, respectively, in order of appearance.

FIG. 9A is a table showing determination results of genotypes and the presence or absence of mutations in Test Example 5 in a case of using a method of the present invention.

FIG. 9B shows images of results of T7EI assay on blastocysts of ten embryos in Test Example 5 by using the method of the present invention.

FIG. 9C is a table showing determination results of genotypes and the presence or absence of mutations in Test Example 5 in a case of using a method of the related art as a control group.

FIG. 10A is a schematic diagram showing a protocol in Test Example 6.

FIG. 10B is a schematic diagram showing a sequence of a guide RNA used for modification of Ggat1 gene in Test Example 6. FIG. 10B discloses SEQ ID NO: 108.

FIG. 10C shows images of results of T7EI assay on fibroblasts in Test Example 6.

FIG. 10D is a graph showing results of FACS analysis utilizing the fact that FITC-labeled isolectin, BS-I-B4 (IB4) specifically recognizes α-Gal epitope in Test Example 6.

FIG. 10E is an image in which fibroblasts are imaged after a survival assay by treatment with IB4-saporin conjugate (IB4SAP) in Test Example 6.

FIG. 11A is a schematic diagram showing a protocol in Test Example 7.

FIG. 11B is a schematic diagram showing a sequence of a guide RNA used for modification of Alb1 gene in Test Example 7. FIG. 11B discloses SEQ ID NO: 109.

FIG. 11C is an image showing results of T7EI assay on EGFP-positive or to EGFP-negative hepatocytes of a Cas9 mouse or a wild-type mouse in Test Example 7.

FIG. 12A is a table showing indel mutation ratios of 12.5- to 13-day-old fetuses in Test Example 8.

FIG. 12B is an image showing the jaw formation state of 12.5- to 13-day-old endothelin 1 (ET-1) gene knockout phenotype mouse fetus and wild-type mouse fetus in Test Example 8.

FIG. 12C is a graph showing mosaic ratios of fetuses derived from each fertilized egg in Test Example 8.

FIG. 13 is a graph showing mosaic ratios of fetuses derived from each of fertilized eggs having different introduction times of a guide RNA in Test Example 9.

FIG. 14 is a graph showing indel mutation ratios and mosaic ratios of fetuses derived from each of fertilized eggs having different introduction concentrations of a guide RNA in Test Example 10.

FIG. 15A is a schematic diagram showing a sequence of a knock-in fragment and a guide RNA used for modification of genes in Test Example 11. FIG. 15A discloses SEQ ID NO: 107.

FIG. 15B is an image showing a result of subjecting a PCR product obtained by using Klf5 gene as a template in PCR, to agarose gel electrophoresis by using a solution prepared from a blastocyst derived from a fertilized egg of a Cas9 mouse in Test Example 11.

FIG. 15C is a table showing knockin efficiency in a blastocyst derived from a fertilized egg of a Cas9 mouse or a wild-type mouse in Test Example 11.

### DESCRIPTION OF EMBODIMENTS

<Genetic Modification Non-Human Organism>

As one embodiment, the present invention provides a genetic modification non-human organism in which a nuclear genome includes at least 3 copies of genes that code for Cas9 (CRISPR-associated 9).

In the genetic modification non-human organism of the present embodiment, Cas9 is overexpressed in all or any one of cells. According to the inventors of the present invention, it became clear that an overexpressed state of Cas9 does not affect the body of the non-human organism. In addition, because Cas9 is overexpressed in advance, it is possible to simply and rapidly modify target genes by only introducing a guide RNA.

In the present specification, the term “genetic modification” means performing of specific gene disruption, knockin of a reporter gene, and the like by carrying out recombination of targeted gene or targeted mutation (for example, insertion of SNP, partial mutation, and the like) through techniques such as CRISPR/Cas9 system, Transcription Activator-Like Effector Nucleases (TALEN), and the like.

In the present specification, examples of the “non-human organism” include, but are not limited to, E. coli, Bacillus subtilis, yeast, insect, plant (such as monocotyledon or dicotyledon), bird animals such as chickens, mammals such as mice, rats, hamsters, guinea pigs, rabbits, dogs, cats, horses, cows, sheep, pigs, goats, marmosets, and monkeys. Among these, mammals are preferable, and rodents such as mice, rats, guinea pigs, hamsters, and rabbits are preferable.

Examples of mice include C57BL/6 strain, DBA2 strain, and the like as pure strains, include B6C3F1 strain, BDF1 strain, B6D2F1 strain, BALB/c strain, ICR strain, and the like as hybrid strains, but the examples are not limited thereto. Specific examples of rats include Wistar, SD, and the like, but the examples are not limited thereto.

[Cas9]

“Cas9” is one of the Cas protein families constituting the adaptive immune system that provides acquisition resistance against invading foreign nucleic acid in bacteria and archaebacteria and is an endonuclease that recognizes a PAM sequence in exogenous invading DNA so as to cleave the double-stranded DNA to produce upstream blunt ends. In the present specification, Cas9 means an enzyme that forms a complex with the guide RNA and has DNA cleavage activity.

In the present specification, examples of Cas protein families include Cas1, Cas1B, Cas2, Cas3, Cas4, Cas5, Cas6, Cas7, Cas8, Cas9 (known as Csn1 and Csx12), Cas 10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, modified ones thereof, and the like. Among these, it is particularly preferable that Cas9 genes, homologues thereof, or modified ones thereof be introduced into the genetic modification non-human organism of the present embodiment.

Cas9 directs cleavage of one or both strands within base pairs of, for example, 1, 2, 3, 4, 5, 6, 7, 8, 9, 10, 15, 20, 25, 50, 100, 200, 500 or more, from the first or last nucleotide of the PAM sequence in a target gene. Which of bp upstream of the PAM sequence is to be cleaved depends on bacterial species derived from Cas9, but most of Cas9 including the one from Streptococcus pyogenes (S. pyogenes) cleaves three bases upstream of the PAM sequence.

In addition, Cas9 used in the present embodiment may be a variant lacking activity of cleaving one or both strands of a target gene containing a target sequence. For example, substitution from aspartic acid in a RuvCI catalytic domain of Cas9 derived from S. pyogenes to alanine (D10A) converts Cas9 from nuclease which cleaves both strands to nickase (which cleaves one strand). Other examples of mutations in which Cas9 is converted to nickase include, but are not limited to, H840A, N854A, and N863A. In addition, two or more catalytic domains of Cas9 (RuvCI, RuvCII, and RuvCIII) can produce mutated Cas9 in which entire DNA cleavage activity is substantially lost. In this case, Cas9 is preferably other chimeric enzymes fused with an enzyme having DNA cleavage activity. The D10A mutation may be combined with one or a plurality of H840A, N854A, or N863A mutations so as to produce the Cas9 in which entire DNA cleavage activity is substantially lost. In a case where the DNA cleavage activity of the mutated Cas9 is less than about 25%, 10%, 5%, 1%, 0.1%, or 0.01% with respect to a non-mutated form thereof, entire DNA cleavage activity is regarded to be substantially lost.

In the present embodiment, a base sequence that codes for Ca9 may be codon-optimized for expression in specific cells such as eukaryotic cells. Examples of eukaryotic cells include, but are not limited to, specific organisms such as humans, mice, rats, rabbits, dogs, pigs, or nonhuman primates. In general, codon optimization refers to a process in which while maintaining native amino acid sequences, at least one codon of the native sequence is replaced with a codon that is used more frequently or most frequently in organism genes to be introduced, and thus a nucleic acid sequence is modified for enhanced expression in the organism genes to be introduced. Various species exhibit a specific bias for a specific codon of a specific amino acid. The codon bias (difference in frequency of occurrence of codon usage between organisms) is often correlated with the translation efficiency of mRNA, and it is considered that this depends especially on characteristics of the codon being translated and the availability of a specific tRNA. The predominant selected tRNAs in cells is generally a reflection of the most frequently used codons in peptide synthesis. Accordingly, genes can be personalized for optimal gene expression in a predetermined organism based on codon optimization. The frequency of occurrence of codon usage is readily available in the “Codon Usage Database” posted on www.kazusa.or.jp/codon/(visited on Dec. 16, 2015), and by using this table, it is possible to optimize the codon (for example, refer to “Codon usage tabulated from the international DNA sequence databases: status for the year 2000”, Nucl. Acids Res. 28:292 (2000) by Nakamura, Y, et al. Computer algorithms for codon optimization of specific sequences for expression in a specific organism species are also available, for example, in Gene Forge (Aptagen, LLC.; Jacobus, Pa.), and the like. In the present embodiment, one or a plurality of codons in the sequence that codes for Cas9 corresponds to the codon most frequently used for a specific amino acid.

As an expression level of Cas9 cells increases, it is not necessary to introduce Cas9 from the outside, and therefore a large amount of guide RNA can be introduced. Therefore, it is possible to efficiently carry out genetic modification on one location or a plurality of locations with respect to one gene by using the CRISPR system, and it is also possible to modify not only one gene but also a plurality of genes at the same time by a single operation. In the present embodiment, it is preferable that the copy number of the Cas9 gene introduced be 3 or more. This is because that in general, as the copy number of the introduced Cas9 gene increases, an expression level of Cas9 tends to increase, although an expression level is also influenced by a locus into which the Cas9 gene is introduced, and a promoter. The copy number of a gene that codes for the introduced Cas9 may be 3, 4, 5, 6, 7, 8, 9, 10, 20, 50 or more, for example.

In the present specification, the term “nuclear genome” means all chromosomes contained in the nucleus. For example, in a case of humans, the number of chromosomes constituting the nuclear genome is a total 46 of 22 pairs of autosomal chromosomes and two sex chromosomes. In a case of bovines, the number of chromosomes is a total of 60 of 29 pairs of autosomal chromosomes and two sex chromosomes. In a case of mice, the number of chromosomes is a total of 40 of 19 pairs of autosomal chromosomes and two sex chromosomes.

In the present specification, the term “copy number” means the number of base sequences of a specific gene present in the nuclear genome. For example, in a case where one specific gene is present on one chromosome constituting the nuclear genome, the copy number is 1. In addition, in a case where one specific gene is present on one chromosome and a homologous chromosome thereof (that is, in a case of a homozygote), the copy number is 2.

In the present embodiment, a plurality of Cas9 genes may be introduced into the same chromosome, and one or a plurality of Cas9 genes may be introduced into different chromosomes. In addition, the Cas9 gene may be tandemly introduced into the same locus on the same chromosome.

In the present embodiment, a locus into which the Cas9 gene is introduced is a gene region in which the expression is constantly and stably performed and is a region that enables the survival of life even in a case where a gene originally coded in this region is lost or modified. In addition, in a case of utilizing the CRISPR system in a case where the Cas9 gene is introduced, it is preferable to have a PAM sequence in the vicinity. Examples of the locus into which the Cas9 gene is introduced include GTP-binding protein 10 locus, Rosa26 locus, beta-actin locus, and the like.

[Promoter]

In the present embodiment, the Cas9 gene introduced into the nuclear genome may contain a promoter operably linked to the Cas9 gene. Examples of the promoter include a viral promoter, an expression-inducible promoter, a tissue-specific promoter, or a promoter obtained by fusing an enhancer sequence or a promoter sequence, and the like. It is preferable that the promoter be linked to upstream (5′ side) of the Cas9 gene.

In the present specification, the term “viral promoter” means a virus-derived promoter. Examples of viruses from which a promoter is derived include cytomegalovirus, moloney leukemia virus, JC virus, mammary tumor virus, simian virus, retrovirus, and the like.

In the present specification, the term “expression-inducible promoter” means a promoter by which the expression of a gene (in the present embodiment, the Cas9 gene) to be expressed in a case where a specific stimulus such as a chemical agent or physical stress is given is caused and expression activity is not caused in the absence of stimulation. Examples of the expression-inducible promoter include, but not are limited to, a TetO (tetracycline operator) promoter, a metallothionein promoter, IPTGA/lacI promoter system, ecdysone promoter system, “lox stop lox” system by which an inhibitory sequence is irreversibly lost during translation or transcription, and the like.

In the present specification, the term “tissue-specific promoter” means a promoter which exhibits activity only in a specific tissue. Examples of the tissue-specific promoter include, but are not limited to, a tyrosinase promoter or a TRP2 promoter in a case of melanoma cells or melanocytes; a MMTV promoter or a WAP promoter in a case of breast cells or breast cancer; a villin promoter or an FABP promoter in a case of intestinal cells or intestinal cancer; a PDX promoter in a case of pancreatic cells; a RIP promoter in a case of pancreatic beta cells; a keratin promoter in a case of keratinocytes; a probasin promoter in a case of prostate epithelium; a nestin promoter or a GFAP promoter in a case of cells of the central nervous system (CNS) or cancers of the central nervous system; a tyrosine hydroxylase promoter, an S100 promoter, or a neurofilament promoter in a case of neurons; a pancreas-specific promoter described in Edlund et al., Science, 230:912-916 (1985); a clara cell secretory protein promoter in a case of lung cancer; an α-myosin promoter in a case of cardiac cells, and the like.

Examples of a promoter obtained by fusing an enhancer sequence or a promoter sequence include an SR-α promoter including a promoter of early genes of simian virus 40 (SV40) and a partial sequence of the long terminal repeat of human T-cell leukemia virus 1; a CAG promoter including immediate early (IE) gene enhancer of cytomegalovirus and a chicken β-actin promoter; and the like. In particular, the CAG promoter enables overexpression of a gene to be expressed (in the present embodiment, the Cas9 gene) in almost the whole body by providing a poly(A) signal site of a rabbit β-globin gene at the 3′ end of the gene to be expressed (in the present embodiment, the Cas9 gene).

Among these, the viral promoter or the CAG promoter is preferable as a promoter in a case where the gene is desired to be constantly expressed at a high level in the whole body. In addition, in a case where a timing of expression of Cas9 or a tissue to be expressed is adjusted, the expression-inducible promoter or the tissue-specific promoter is preferable.

In the present specification, the phrase “operably linked” means a functional link between a gene expression regulatory sequence (for example, a promoter or a series of transcription factor binding sites) and a gene to be expressed (in the present embodiment, the Cas9 gene). The term “expression regulatory sequence” means a sequence which directs transcription of a gene to be expressed (in the present embodiment, the Cas9 gene).

In the present embodiment, the Cas9 gene introduced into the nuclear genome may be linked to the downstream (3′ side) of the Cas9 gene such that a polyadenylation signal necessary for polyadenylation of the 3′ end of mRNA is operable. As the polyadenylation signal, there is a polyadenylation signal contained in each gene derived from the above viruses or from various human or non-human organisms, for example, a polyadenylation signal of late genes or early genes of SV40, rabbit β-globin genes, bovine growth hormone genes, human A3 adenosine receptor gene, and the like. In addition, in order to further express the Cas9 gene at a higher level, a splicing signal, an enhancer region, a part of introns of each gene may be linked to the 5′ upstream of a promoter region, between the promoter region and a translation region, or to the 3′ downstream of the translation region.

In the present embodiment, in the Cas9 gene introduced into the nuclear genome, one or a plurality of nuclear localization sequences (NLS) may be operably linked to the upstream (5′ side) or the downstream (3′ side) of the Cas9 gene.

Examples of the NLS include, but are not limited to, NLS of the SV40 virus large T antigen having an amino acid sequence PKKKRKV (SEQ ID NO: 1); NLS derived from nucleoplasmin (for example, bisect NLS of nucleoplasmin having a sequence KRPAATKKAGQAKKKK (SEQ ID NO: 2)); c-myc NLS having an amino acid sequence PAAKRVKLD (SEQ ID NO: 3) or RQRRNELKRSP (SEQ ID NO: 4); hRNPA1 M9 NLS having a sequence NQSSNFGPMKGGNFGGRSSGPYGGGGQYFAKPRNQGGY (SEQ ID NO: 5); a sequence RMRIZFKNKGKDTAELRRRRVEVSVELRKAKKDEQILKRRNV (SEQ ID NO: 6) of the IBB domain ofimportin-alpha; sequences VSRKRPRP (SEQ ID NO: 7) and PPKKARED (SEQ ID NO: 8) of fibroid T protein; a sequence PQPKKKPL (SEQ ID NO: 9) of human p53; a sequence SALIKKKKKMAP (SEQ ID NO: 10) of mouse c-ab1 IV; sequences DRLRR (SEQ ID NO: 11) and PKQKKR (SEQ ID NO: 12) of influenza virus NS1; a sequence RKL KKKIKKT (SEQ ID NO: 13) of hepatitis virus delta antigen; a sequence REKKKFLKRR (SEQ ID NO: 14) of mouse M×1 protein; a sequence KRKGDEVDGVDEVAKKKSKK (SEQ ID NO: 15) of human poly(ADP-ribose) polymerase; a sequence RKCLQAGMNLEARKTKK (SEQ ID NO: 16) of steroid hormone receptor (human), glucocorticoid receptor; and the like.

In the present embodiment, in the Cas9 gene introduced into the nuclear genome, one or a plurality of marker genes such as fluorescent protein may be operably linked to the upstream (5′ side) or the downstream (3′ side) of the Cas9 gene. Examples of the fluorescent protein include green fluorescent protein (GFP), yellow fluorescent protein (YFP), blue fluorescent protein (BFP), cyan fluorescent protein, red fluorescent protein, and the like.

[Method for Producing Genetic Modification Non-Human Organism]

The genetic modification non-human organism of the present embodiment can be prepared by using a known genetic modification technique (for example, CRISPR system, a method of using Transcription Activator-Like Effector Nucleases (TALEN), a method of using zinc finger nuclease, and the like) for producing a genetic modification non-human organism in which foreign genes are introduced into the nuclear genome. In addition, the genetic modification non-human organism can be prepared by a method in which the Cas9 gene (preferably, one in a linear state) is introduced into a fertilized egg, an embryonic stem cell, a sperm or an unfertilized egg of a target non-human mammal, and from individuals generated using these cells, an individual in which the Cas9 gene is integrated into the chromosome of all cells containing germinal cells is selected. Among these, the genetic modification non-human organism of the present embodiment can be efficiently obtained by a method for producing a genetic modification non-human organism using the CRISPR system of the related art. More specifically, there is a method described in “Method for Selectively and Site-Specifically Modifying Target Genes” to be described later, and the genetic modification non-human organism can be produced by using a vector containing, as a foreign gene, a gene having a sequence homologous to a locus in which a target gene is inserted into the upstream (5′ side) and the downstream (3′ side) of the Cas9 gene, and the Cas9 gene described above, thereby introducing the gene into the target non-human organism.

In addition, the genetic modification non-human organism of the present embodiment may be a genetic modification non-human organism having a nuclear genome into which a foreign gene other than Cas9 gene is introduced at the same time and which thus becomes the nuclear genome into which the Cas9 gene and the foreign gene other than the Cas9 gene are introduced.

The presence of the Cas9 gene in germinal cells of the obtained genetic modification non-human organism can be confirmed by the fact that the offspring of the obtained organism has transgenes in all of the germinal cells and somatic cells thereof. Selection of individuals can be carried out by confirming at the DNA level that the Cas9 gene is present in genomic DNA prepared from some of tissues constituting the individual such as blood tissue, epithelial tissue, connective tissue, cartilage tissue, bone tissue, muscle tissue, oral tissue, or skeletal tissue.

Alternatively, the presence of the Cas9 gene can be confirmed by a genotyping method using a single blastocyst after the introduction of the Cas9 gene (reference material: Sakurai T., et al., “A single blastocyst assay optimized for detecting CRISPR/Cas9 system-induced indel mutations in mice”, BMC Biotech., 14:69, 2014.). According to this method, whether or not the Cas9 gene is present can be determined more quickly compared to that of the related art.

The individual selected in the above manner is generally a heterozygote having the Cas9 gene on one side of the homologous chromosome, and thus it is possible to obtain a homozygous organism having the Cas9 gene on both sides of the homologous chromosome from the offspring by crossing individuals of the heterozygote. By crossing male with female organisms of this homozygote, all offspring become homozygotes that stably hold the Cas9 gene, and therefore transgenerational reproduction of the genetic modification non-human organism of the present embodiment is possible under general breeding environments.

<Egg Cell>

In one embodiment, the present invention provides an egg cell derived from the genetic modification non-human organism having a nuclear genome into which at least 3 copies of the genes that code for Cas9 are introduced.

In the egg cell of the present embodiment, a plurality of different genes or a plurality of locations in the same gene can be edited at the same time with high efficiency. In addition, according to a fertilized egg obtained by fertilizing the egg cell of the present embodiment and sperm derived from the same species of the organism, it is possible to rapidly produce the genetic modification non-human organism at a low cost.

The egg cell of the present embodiment is derived from a female of the genetic modification non-human organism described above.

In the present specification, the term “egg cell” means a female reproductive cell, including an oocyte and an unfertilized egg.

As the egg cell derived from the genetic modification non-human organism according to the present embodiment, in a case where the genetic modification non-human organism is a heterozygote having the Cas9 gene on one side of a homologous chromosome in the nuclear genome, there is an egg cell having, within the nucleus, the nuclear genome into which at least 3 copies of the Cas9 genes are introduced, and there is an egg cell not having the nuclear genome into which the Cas9 genes are introduced. Any of the egg cells can be utilized to modify a target gene. This is because protein or mRNA of maternally derived Cas9 (hereinafter will be referred to as maCas9) accumulates in the egg cell during the oogenesis and gradually decomposes after functioning to the 2-cell stage from the fertilized egg. Therefore, in a case where it is desired to produce the genetic modification non-human organism that does not have a foreign gene and in which a target gene is modified, it is preferable to use the egg cell not having, within the nucleus, the nuclear genome into which the Cas9 gene is introduced.

It is sufficient that the genetic modification non-human organism from which the egg cell is derived in the present embodiment have the nuclear genome into which at least 3 copies of the Cas9 genes are introduced, and protein or mRNA of maCas9 can accumulate in an oocyte during the oogenesis. In addition, the larger the copy number of the introduced Cas9 gene becomes, the more the accumulated protein or mRNA of maCas9 increases, which is preferable. The copy number of the introduced Cas9 gene may be 3, 4, 5, 6, 7, 8, 9, 10, 20, 50 or more, for example.

<Fertilized Egg>

In one embodiment, the present invention provides a fertilized egg obtained by fertilizing the egg cells derived from the genetic modification non-human organism having the nuclear genome into which at least 3 copies of the genes that code for Cas9 are introduced, and sperm derived from the same species of the organism.

According to the fertilized egg of the present embodiment, a plurality of different genes or a plurality of locations in the same gene can be edited at the same time with high efficiency. In addition, according to the fertilized egg of the present invention, it is possible to rapidly produce the genetic modification non-human organism at a low cost.

The fertilized egg of the present embodiment can be obtained by artificially fertilizing the above-described egg cell derived from a female of the genetic modification non-human organism and sperm derived from the same species of the organism.

As the egg cell derived from the genetic modification non-human organism according to the present embodiment, in a case where the genetic modification non-human organism is a heterozygote having the Cas9 gene on one side of a homologous chromosome in the nuclear genome, there is an egg cell having, within the nucleus, the nuclear genome into which at least 1 copy of the Cas9 gene is introduced, and there is an egg cell not having the nuclear genome into which the Cas9 gene is introduced. Any of the egg cells can be utilized to modify a target gene. This is because protein or mRNA of maCas9 accumulates in the egg cell during the oogenesis and gradually decomposes after functioning to the 2-cell stage from the fertilized egg. Therefore, in a case where it is desired to produce the genetic modification non-human organism that does not have a foreign gene and in which a target gene is modified, it is preferable to use the egg cell not having, within the nucleus, the nuclear genome into which the Cas9 gene is introduced.

It is sufficient that the genetic modification non-human organism from which the egg cell is derived in the present embodiment have the nuclear genome into which at least 3 copies of the Cas9 genes are introduced, and protein or mRNA of maCas9 can accumulate in an oocyte during the nuclear genome oogenesis. In addition, the larger the copy number of the introduced Cas9 gene becomes, the more the accumulated protein or mRNA of maCas9 increases, which is preferable. The copy number of the introduced Cas9 gene may be 3, 4, 5, 6, 7, 8, 9, 10, 20, 50 or more, for example.

Sperm in the present embodiment may be sperm of the same species as the organism from which the egg cell was obtained and may be sperm derived from the genetic modification non-human organism having, within the nucleus, the nuclear genome into which at least one copy of the Cas9 gene is introduced, may be sperm derived from the genetic modification non-human organism not having the nuclear genome into which the Cas9 gene is introduced, or may be sperm derived from a wild-type organism. In a case where it is desired to prepare the genetic modification non-human organism that does not have the Cas9 gene and in which a target gene is modified, the sperm that does not have, within the nucleus, the nuclear genome into which the Cas9 gene is introduced or is derived from a wild-type organism is preferable.

In the egg cell not having, within the nucleus, the nuclear genome into which the Cas9 gene is introduced, protein or mRNA of maCas9 is accumulated, as described above. Therefore, in a case of a fertilized egg obtained by fertilizing the egg cell not having, within the nucleus, the nuclear genome into which the Cas9 gene is introduced, and the sperm that does not have, within the nucleus, the nuclear genome into which the Cas9 gene is introduced or is derived from a wild-type organism, protein or mRNA of maCas9 is transiently present excessively in a short period of the 2-cell stage from the fertilized egg. Accordingly, as shown in examples described later, mosaic mutations become significantly lower than those of a genomic editing method of the related art in which synthetic Cas9 mRNA and guide RNA are microinjected into the fertilized egg.

<Method for Modifying Target Gene>

In one embodiment, the present invention provides a method for modifying a target gene including a step of introducing a guide RNA into the cell derived from the genetic modification non-human organism described above, the egg cell described above, or the fertilized egg described above.

In one embodiment, the present invention further provides a method for modifying a target gene, including a step of introducing the guide RNA into the cell derived from the genetic modification non-human organism described above, the egg cell described above, or the fertilized egg described above; a step of cleaving the target gene at a cleavage site located upstream of a PAM (Proto-spacer Adjacent Motif) sequence by Cas9 expressed in the cell derived from the genetic modification non-human organism, the egg cell derived from the genetic modification non-human organism, or the fertilized egg derived from the genetic modification non-human organism; and a step of obtaining the modified target genes in a region determined by complementary binding of the guide RNA and the target gene, in which the target gene has the PAM sequence, and the guide RNA contains a polynucleotide including a base sequence complementary to an upstream base sequence of the PAM sequence in the target gene.

According to the method of the present embodiment, in vitro and in vivo editing of the target gene can be carried out simply and efficiently. In addition, the genetic modification non-human organism can be rapidly produced at a low cost.

In the present specification, the “target gene” is not particularly limited as long as the target gene is a gene having the PAM sequence, and examples thereof include a gene that can be studied as a gene related to lifestyle diseases such as cancer or heart disease.

In the present specification, the “PAM sequence” is a sequence recognizable by Cas9, and a length and a base sequence of the PAM sequence vary depending on the bacterial species from which Cas9 is derived. For example, S. pyogenes recognizes three bases of “NGG” (N represents any base).

Streptococcus thermophiles (S. thermophilus) has two Cas9s and recognizes 5 and 6 bases of “NGGNG” or “NNAGAA” (N represents any base) as a PAM sequence, respectively. It is preferable that the PAM sequence be “NGG”, because the PAM sequence to be recognized is short and the editable target gene is unlikely to be limited.

In the present embodiment, it is sufficient that Cas9 expressed in the cell derived from the genetic modification non-human organism, the egg cell derived from the genetic modification non-human organism, or the fertilized egg derived from the genetic modification non-human organism recognize the PAM sequence, “NGG”, and examples thereof include Cas9 derived from S. pyogenes, and the like. In addition, as described in the section of “Genetic Modification Non-Human Organism” described above, Cas9 may be modified, or may be one obtained by expressing a Cas9 gene including a base sequence in which a codon is optimized.

In the present specification, the “guide RNA” is one that mimics a hairpin structure of a tracrRNA-crRNA chimera obtained by fusing a small RNA fragment (CRISPR-RNA: crRNA) containing a foreign sequence (guide sequence) and constituting the adaptive immune system that provides acquisition resistance against invading foreign nucleic acid in bacteria and archaebacteria, and RNA that is partially complementary to the crRNA (trans-activating crRNA: tracrRNA).

The guide RNA used in the present embodiment may be a tracrRNA-crRNA chimera synthesized in a state where crRNA containing the guide sequence and tracrRNA are fused or may be a tracrRNA-crRNA chimera in which crRNA containing the guide sequence and tracrRNA are separately produced and annealed before introduction.

The guide RNA in the present embodiment contains, at the 5′ end region, a polynucleotide composed of a base sequence complementary to a base sequence from 1 base upstream of the PAM sequence in the target gene, to preferably 20 bases to 24 bases, and to more preferably 22 bases to 24 bases. Furthermore, the guide RNA may contain one or more polynucleotides which have a base sequence non-complementary to the target gene and is composed of a base sequence that is arranged in a symmetrical and complementary manner with one point as an axis and that can have a hairpin structure.

The method of the present embodiment will be described in detail below.

[Introduction Step]

First, guide RNAs are introduced into cells derived from the genetic modification non-human organism, the egg cells described above, or the fertilized eggs described above. As a method for introducing the guide RNAs, known gene introduction methods (for example, calcium phosphate method, electroporation method, lipofection method, coagulation method, microinjection method, particle gun method, DEAE-dextran method, and the like) can be used. Among these, the electroporation method is preferable because the operation is simple. In the genetic modification method utilizing the CRISPR system of the related art, the member of Cas9 mRNA was large and an introduction efficiency was poor. However, in the method of the present embodiment, the cells derived from genetic modification non-human organism in which Cas9 can be expressed, the egg cells, or fertilized eggs are used, and therefore it is possible to easily and efficiently introduce the guide RNAs by the electroporation method and the like.

In a case where an introduction target is a cell derived from the genetic modification non-human organism, the guide RNA can be introduced into a living genetic modification non-human organism. In this case, the introduction method may be a method known to those skilled in the art, such as intra-arterial injection, intravenous injection, subcutaneous injection, intranasal introduction, transbronchial introduction, intramuscular introduction, percutaneous introduction, or oral introduction, and the intravenous injection is preferable.

In addition, in a case where a tissue-specific promoter is operably linked to the upstream (5′ side) of the Cas9 gene, by the guide RNA being directly introduced into the entire body of the genetic modification non-human organism or target tissues, the genetic modification is performed only in the target tissues by the guide RNA and Cas9, and therefore the dynamics of diseases can be observed.

In a case where the introduction target is a fertilized egg fertilized by the above egg cell, or a fertilized egg, it is preferable to use a fertilized egg of 4 hours to 24 hours, preferably 6 hours to 12 hours, and more preferably 6 hours to 8 hours after fertilization, in which male pronucleus is developed. With the time after fertilization being within the above range, an expression level of protein or mRNA of maCas9 becomes high, and an embryo of a so-called mosaic-type in which both the cell in which the target gene is modified and the cell in which the target gene is not modified are present, is prevented from being generated. Therefore, it is possible to modify the target gene with high efficiency.

In addition, as described in the above section, “Fertilized Eggs”, in a case where it is desired to produce the genetic modification non-human organism which does not have, within in the nucleus, the nuclear genome into which the Cas9 gene is introduced, and in which the target gene is modified, a fertilized egg to be used is preferably a fertilized egg obtained by fertilizing an egg cell not having, within the nucleus, the nuclear genome into which the Cas9 gene is introduced, and sperm derived from an organism not having, within the nucleus, the nuclear genome into which the Cas9 gene is introduced, or a wild-type organism. In the egg cell not having, within the nucleus, the nuclear genome into which the Cas9 gene is introduced, protein or mRNA of maCas9 is accumulated. Therefore, in a case where the fertilized egg to be used is a fertilized egg obtained by fertilizing an egg cell not having, within the nucleus, the nuclear genome into which the Cas9 gene is introduced, and sperm derived from an organism not having, within the nucleus, the nuclear genome into which the Cas9 gene is introduced, or a wild-type organism, protein or mRNA of maCas9 is transiently present excessively in a short period of the 2-cell stage from the fertilized egg. Accordingly, as shown in the examples described later, mosaic mutations become significantly lower than those of the genomic editing method of the related art in which synthetic Cas9 mRNA and guide RNA are microinjected into the fertilized egg.

In a case of introducing the guide RNA into the fertilized egg, a concentration of the guide RNA is not particularly limited, but as the concentration becomes higher, genetic modification can be performed with higher probability. A concentration of the guide RNA may be, for example, 1 ng/μL to 300 ng/μL.

In addition, in a case where the fertilized egg is derived from the genetic modification non-human animal (particularly, a non-human mammal), after introduction of the guide RNA, the fertilized egg is implanted to the uterus or the oviduct of the corresponding non-human animal so as to be generated, and therefore it is possible to easily obtain the genetic modification non-human animal.

In a case where two or more locations of one target gene are modified, or in a case where two or more of genes are used as target genes, two or more types of the guide RNAs containing, in the 5′ end region, a polynucleotide including a base sequence complementary to the base sequence of each gene, may be designed so as to be introduced. Accordingly, it is possible to edit a plurality of different genes or a plurality of locations in the same gene at the same time with high efficiency.

In the method of the present embodiment, a foreign gene may be introduced together with the guide RNA. By introducing a foreign gene together, it is possible to obtain the genetic modification non-human organism having the nuclear genome in which after cleavage of a target gene, the foreign gene is inserted into the target gene by homologous recombination.

In addition, it is preferable that the foreign gene not have a PAM sequence. In the foreign gene, for example, in a case where the PAM sequence is changed to a non-PAM sequence by one base substitution not changing the amino acid sequence, and the like, it is possible to insert the foreign gene on the nuclear genome by homologous recombination and it is also possible to prevent the foreign gene from being cleaved again by the endogenous Cas9.

It is preferable that the foreign gene be introduced using a vector containing the foreign gene. In the vector containing the foreign gene, it is preferable that a DNA having a sequence homologous to a site into which a target gene is insert be linked to the 5′ end and the 3′ end of the foreign gene. The number of bases of the DNA having a sequence homologous to a site into which a target gene is inserted is preferably 100 bases to 300 bases. In the vector containing the foreign gene, the promoter, the polyadenylation signal, the NLS, the fluorescent protein marker gene, which are described in the above section, “Genetic Modification Non-Human Organism”, and the like may be operably linked to the 5′ end or the 3′ end of the foreign gene.

In a case of use in the present specification, the “vector” is a tool that enables or facilitates the transfer of entities from one environment to another environment. For example, some vectors used in recombinant DNA technology enable entities such as segments of DNA (for example, heterologous DNA segments such as heterologous cDNA segments) to be transferred into non-human organism cells. In the present embodiment, the vector includes viral vectors (for example, lentiviral vectors, baculovirus vectors, adenovirus/adeno-associated viral vectors, and the like), bacterial vectors, protozoal vectors, DNA vectors, or recombinant vectors which may contain recombination thereof.

[Cleavage Step]

Cas9 expressed in the cells derived from the genetic modification non-human organism, the egg cell derived from the genetic modification non-human organism, or the fertilized egg derived from the genetic modification non-human organism, and the guide RNA are combined in mild conditions in vitro or in vivo so as to form a Cas 9-guide RNA complex. The mild conditions indicate a temperature and a pH at which a protein does not decompose or denature, and the temperature may be 4° C. to and 40° C. and the pH may be 4 to 10. Accordingly, the Cas9-RNA complex can be formed even in the body of the living genetic modification non-human organism.

In the Cas9-RNA complex, some of the guide RNA binds to the target gene and Cas9 recognizes the PAM sequence in the target gene. Subsequently, the target gene is cleaved at a cleavage site located 3 bases upstream so as to produce blunt ends. More specifically, Cas9 recognizes the PAM sequence, a double helix structure of the target gene is peeled from the PAM sequence as a starting point, followed by annealing with the base sequence complementary to the target gene in the guide RNA, and therefore the double helix structure of the target gene is partially loosened. At this time, Cas9 cleaves the phosphodiester bond of the target gene at the cleavage site located 3 bases upstream of the PAM sequence so as to create blunt ends.

[Modification Step]

Subsequently, in a region determined by complementary binding between the guide RNA and the target gene, it is possible to obtain a target gene subjected to the modification according to the purpose.

In the present specification, the term “modification” means that the base sequence of the target gene is changed. Examples of thereof include a change in the base sequence of a target double-stranded polynucleotide by cleavage of a target gene and insertion of an exogenous sequence after the cleavage (physical insertion or insertion by replication via homology directed repair), a change in the base sequence of a target gene by non-homologous end joining (NHEJ: DNA ends generated by cleavage are bonded again) after the cleavage, and the like. By the modification of the target gene in the present embodiment, introduction of mutation into the target gene or destruction of a function of the target gene is possible. Therefore, in the present embodiment, in a case of using the fertilized egg, a genetic modification non-human organism in which a function of the target gene is destroyed (knockout) or substituted (knockin) can be easily produced.

<Application and Utilization Method>

In one embodiment, the present invention provides a composition and a method for performing genetic modification. In contrast to previously known methods for targeted genetic recombination, the present invention enables efficient and inexpensive implementation and thus is adaptable to any cell or organism. Any segment of the target gene of a cell or an organism can be modified by the method of the present invention. This method utilizes both a homologous recombination process in which all cells are endogenous and a non-homologous recombination process.

In addition, in one embodiment, the present invention provides a method for performing targeted DNA insertion or targeted DNA deletion. This method includes a step of transforming a cell using a nucleic acid construct having a donor DNA. The scheme concerning the DNA insertion or the DNA deletion after cleavage of a target gene can be determined by those skilled in the art according to known methods.

In addition, in one embodiment, the present invention is utilized in both somatic cells and reproductive cells and provides genetic manipulation at a specific locus.

In addition, in one embodiment, the present invention provides a method for breaking genes in somatic cells. The gene overexpresses products harmful to cells or organisms and expresses products harmful to cells or organisms. Such a gene can be overexpressed in one or more cell types that arise in diseases. Disruption of the overexpressed gene according to the method of the present invention may lead an individual suffering from a disease caused by the overexpressed gene to be healthier.

That is, disruption of only a small proportion of the genes of the cells works and an expression level thereof is reduced, obtaining a therapeutic effect.

In addition, in one embodiment, the present invention provides a method for breaking a gene in reproductive cells. A cell in which a specific gene is disrupted can be selected so as to produce an organism not having a function of the specific gene. In the cell in which the gene is disrupted, the gene can be completely knocked out. A deficiency in a function of this specific cell may have a therapeutic effect.

In addition, in one embodiment, the present invention further provides insertion of a donor DNA that codes for a gene product. This gene product has a therapeutic effect in a case of constitutive expression.

For example, in a population of pancreatic cells, in order to cause the insertion of a donor DNA that codes for an active promoter and an insulin gene, there is a method in which the donor DNA is inserted into an individual suffering from diabetes. The population of pancreatic cells containing exogenous DNA can then produce insulin and thus can treat the individual suffering from diabetes.

In addition, the donor DNA can be inserted into crops and can cause the production of drug-related gene products. Genes of protein products (such as insulin, lipase, and hemoglobin) can be inserted into plants together with regulatory elements (constitutively active promoter or inducible promoter) so as to produce large quantities of drug medicine in plants. Such protein products can then be isolated from plants. Genetic modification plants or genetic modification organisms can be produced by a method using a nucleic acid transfer technique (McCreath, K. J., et al. (2000) Nature 405: 1066-1069; Polejaeva, I. A., et al., (2000) Nature 407: 86-90). Tissue-specific vectors or cell-specific vectors can be utilized to provide gene expression only in selected cells.

In addition, in one embodiment of the present invention, it is possible to select cells that produce cells which are utilized in reproductive cells and in which insertion is performed in a designed manner and all subsequent cell divisions have designed genetic modification.

In addition, in one embodiment, the present invention can be applied to all organisms, cultured cells, cultured tissues, and cultured nuclei (including cells, tissues, or nuclei that can be used to regenerate intact organisms), and gametes (for example, eggs or sperm at various stages of their development).

In addition, in one embodiment, the present invention can be applied to cells derived from any organism (including, but are not limited to, insects, fungi, rodents, cows, sheep, goats, chickens, and other agronomically important animals, and other mammals (including, but are not limited to, dogs, cats, and humans).

In addition, in one embodiment, the present invention can be applied in the production of animal disease models (for example pathological model mice).

In addition, in one embodiment, the present invention can be used in plants. The plants are not particularly limited, and the present invention can be used in any of various plant species (such as monocotyledon or dicotyledon).

### EXAMPLES

Hereinafter, the present invention will be described in further detail with reference to examples and the like, but the present invention is not limited to these examples and the like.

[Experimental Material]

Base sequences of guide RNAs and primers used in the present example are shown in Tables 1 to 3. In Table 1, bold letters (three bases on the 3′ side of a base sequence) indicate base sequences homologous to a PAM sequence on a target gene.

TABLE 1
Guide RNA

| ID | Target Sequence (5′ > 3′) | Location | Gene Bank# | SEQ ID NO |
|---|---|---|---|---|
| Alb-gRNA | CGCAGATGACAGGGTAAGGAAGG | Near Alb exon 7 | NM009654 | 18 |
| Amy-gRnA | ATATGTATTCGATCCCACGTTGG | Amy exon 2 | NM010491 | 19 |
| Crlr-gRNA | ATGTGATCCGTTGGCAACTTAGG | Crlr exon 6 | NM018782 | 20 |
| Et-l-gRNA | GCGCGTCGTACCGTATGGACTGG | Et-1 exon 3 | NM010104 | 21 |
| Ggatl-gRNA | GAGAAAATAATGAATGTCAAAGG | Ggtal exon 4 | NM001145821 | 22 |
| Hprt-gRNA | GGGACTGCGGGTCGGCATGACGG | Hprt exon 1 | NM013556 | 23 |
| Imdn-gRNA | GGCTGCCAGACAACGTGCCGGGG | Imdn exon 2 | NM182928 | 24 |
| Klf5-gRNA | GGTCAGCACCCGCGTGGGCATGG | Klf5 exon 1 | NM009769 | 25 |
| R1-gRNA | GCTCTGCTTGCCATGGCCCCGGG | Ramp1 exon 1 | NM001168392 | 26 |
| R2-gRNA | GGTGAGCACCGTGTCGGGCCCGG | Near Raxnp2 exon 1 | NM019444 | 27 |

TABLE 2
Primer 1

| | | SEQ |
|---|---|---|
| ID | Sequence (5′ → 3′) | ID NO |
| Amy-1S | CGGTGAAGGGTGTTGTGTGA | 28 |
| Amy-1A | ACACAGTCATCAAGCACAAGC | 29 |
| Alb-7S | CTAACGCCTGCTGGTGGTCACAGT | 30 |
| Alb-7A | TGTCTAGCCCCTGCTTCTCT | 31 |
| CAG1620-S | GCTCTAGAGCCTCTGCTAACCATGT | 32 |
| CAGGS-A | GCGGAACTCCATATATGGGCTATGAACTAATG | 33 |
| Cas9 215-A | CGCCGTGCTGTTCTTTTGAGC | 34 |
| Cas9ATG-S | GCCACCATGGACAAGAAGTACTCCATTG | 35 |
| Cas9Cla-A | CCATCGATTCACACCTTCCTCTTCTTCT | 36 |
| Cas9E-S | AAGCGAATTCTCCAAAAGAGTGAT | 37 |
| Crlr-5S | AGAGTTTGCTGAGGCAGGTC | 38 |
| Crlr-5A | CCAGCCCATGTCTCTTCCTA | 39 |
| Et-1-1S | TGGTGTGGCTGTAGTCCTTC | 40 |
| Et-I-1A | CAAGTCAAAGGGGCCTCCAC | 41 |
| Ggtal-S | TCAACCACACAGCTGTTTCTC | 42 |
| Ggtal-A | CTGGCACCAGTCACAGGGAATG | 43 |
| Gtpbp10-S | GTTGTTATAATCTTCTAACAG | 44 |
| Gtpbp10-A | CTGGGTGGAACAATTGATCGT | 45 |
| Hprt-1S | TGAGCCATTGCTGAGGCG | 46 |
| Hprt-1A | CGCGCCTGATCCTTCCTG | 47 |
| Hprt-SF | TGTTGGATACAGGCCAGACTTTGTT | 48 |
| Hprt-SR | TCTTAGGCTTTGTATTTGGCTTTTC | 49 |
| Imdn-1S | ACGCAGGTACCAACCAATCT | 50 |
| Imdn-1A | CCCGGGTTCTTCCCTTATGT | 51 |
| Klf5-1S | GGTACGCGCTCTCTTAGGTT | 52 |
| Klf5-1A | GCGTGTTTCAGATCGTCTCC | 53 |

TABLE 3
Primer 2

| ID | Sequence (5′ > 3′) | SEQ ID NO |
|---|---|---|
| Ramp1-2S | GTCAGAGCGAGGTGCTGAGT | 54 |
| Rampl-2A | CCCATCACCACTCTACTGTCTTG | 55 |
| Ramp1-SF | GCACTGGTGGTCTGGAGGA | 56 |
| Rampl-SR | CCCTCATCACCTGGGATACCT | 57 |
| Ramp2-SF | TGTGCCTCCCTCCGCTGTT | 58 |
| Ramp2-SR | TTACTCCTCCACACCACAAGA | 59 |
| Ramp2-5S | CCGAGCTGGAAGCGAGAG | 60 |
| Ramp2-6A | GACCTCTCCGTCCGGTCT | 61 |
| Ramp3-S | TCGAATTCATCTTAGTTGGCCATGAAGAC | 62 |
| Ramp3-A | TCGAATTCACAGCAGCCGATCAGTGTGCTTG | 63 |
| Sp#2 | GTGGCTGAATGAGACTGGTGTCGAC | 64 |
| Sp-Bottom | CGAAGAGTAACCGTTGCTAGGAGAGACCGTGGCTGAATGAGACTGGTGTCGACACTAGTGG | 65 |
| Sp-EV Top | CCACTAGTGTCGACACCAGTCTCAATTTTTTTTTTCAAAAAAA | 66 |
| NFL (D10A) S | CTGCAGGAATTCGCCACCATGGGACCTAAG | 81 |
| NFL (D10A) A | GCTGTTTGTGCCGATAGCCAGTCCAATAGAG | 82 |

TABLE 4
Off-target analytical primer

| Gene | | ID | Sentence (5′ → 3′) | SEQ ID NO |
|---|---|---|---|---|
| Ramp1 | Ccdc3 | OfftR1-11 | TATATCTGTTTTCCAACCCCAGCTC | 67 |
| | (NM_028804} | OfftR1-12 | TGGGGTACACGTAGACCTTTTATGA | 68 |
| | Trappc13 | OfftR1-21 | TCTCTGGCTGAATTGTTTTACACAGG | 69 |
| | (NM_001093760) | OfftR1-22 | AAAGCAGTCAGCGAACCTTTTTG | 70 |
| | Krt5 | OfftR1-31 | ATGTCTCGCCAGTCCAGTGTGTC | 71 |
| | (NM_027011) | OfftR1-32 | CACCACCAAAACCAAATCCACTG | 72 |
| Ramp2 | Chpf | OfftR2-11 | ACTCAGACGTCTTCGCACCTGTC | 73 |
| | (NM_001001565) | OfftR2-12 | CGCCACATAGTCGGAGTTGTAGA | 74 |
| | B3gat1 | OfftR2-21 | CACCTCTGCAGATGAGGGAAGTG | 75 |
| | (NM_029792) | OfftR2-22 | GGGCATCACCTCGCAGCTTATAG | 76 |
| | SemaSb | OfftR2-31 | CGCCTTTGACATGAACCAAGTG | 77 |
| | (NM_001130456) | OfftR2-32 | ACTCACTGTAGCCTCGGGACCTC | 78 |

Furthermore, the guide RNA was prepared by the following method.

First, using a pgRNA-GFP-T1 vector (#41819, manufactured by Addgene) as a template, a guide RNA to which a TTTTTT site was added at the 5′ end was amplified by PCR. Subsequently, the guide RNA was inserted into a pBluescript II vector (manufactured by Agilent Technologies), and therefore a pBS-T7-gRNA plasmid was obtained. A base sequence of the guide RNA on the obtained plasmid was confirmed by sequencing.

Subsequently, by using MEGAshortscript (registered trademark) T7 kit (manufactured by Life Technologies), the guided RNA was prepared from the plasmid linearized with EcoRI.

### [Example 1] Establishment of Cas9-Highly Expressing Mouse Line

(1) Design of Construct for Inserting Cas9 Gene

Using, as a template, phCas9 (manufactured by Addgene) containing a Cas9 gene (base sequence: SEQ ID NO: 17) in which a codon was optimized by gene synthesis, PCR was carried out using a Cas9ATG-S primer and a Cas9Cla-A, and therefore an amplified gene product was obtained. Subsequently, by PCR, a SV40 nuclear localization signal (NLS) and a base sequence of a Flag tag, or the base sequence of only the Flag tag was inserted immediately after a start codon (ATG) of a Cas9 gene. Therefore, NFCas9 (Cas9 gene having the NLS and the Flag tag) and FCas9 (Cas9 gene having only the Flag tag) were obtained.

Subsequently, each of NFCas9 and FCas9 was inserted into the EcoRI site of a pCAGGS vector so as to construct pCAG-NFCas9 pA and pCAG-FCas9 pA, respectively.

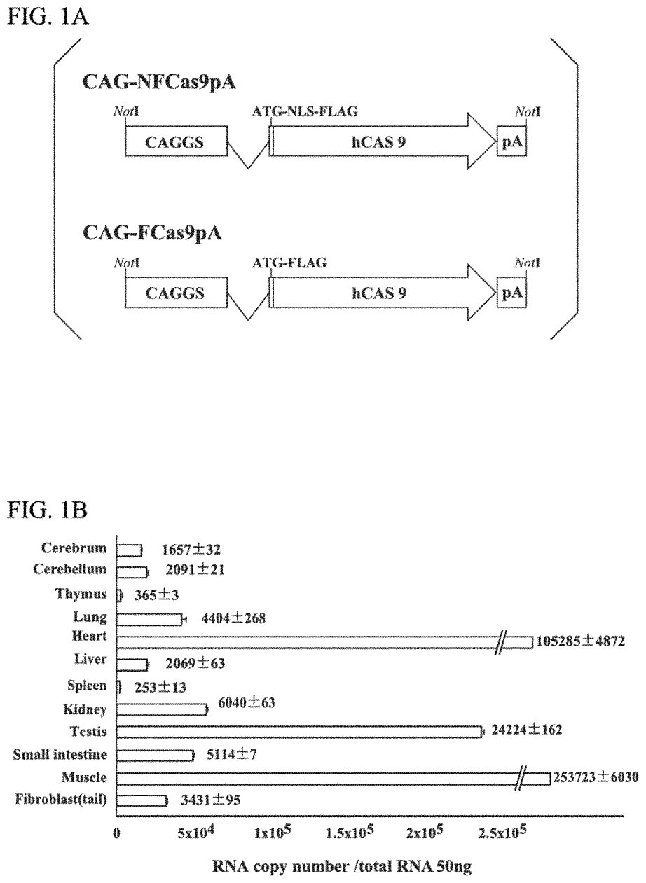

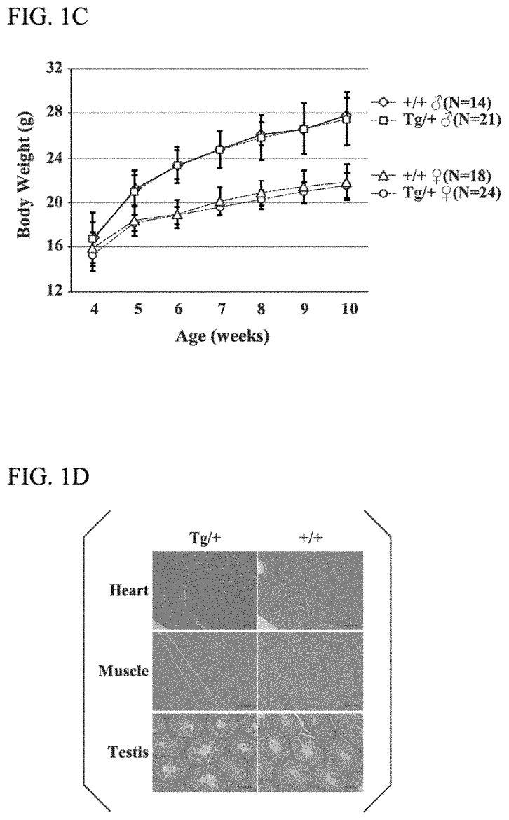

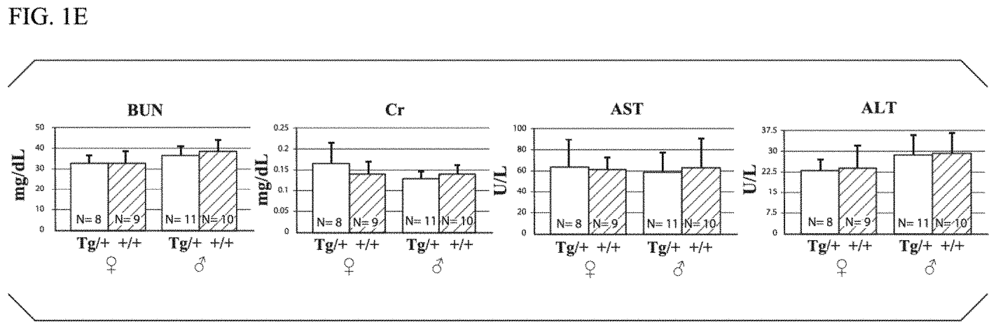

These were each linearized with NotI, and therefore CAG-NFCas9 pA and CAG-FCas9 pA sites were purified (refer to FIG. 1A).

(2) Injection into Fertilized Mouse Egg

In the present example, mice of BDF1 strain, C57BL6/J-Jcl strain (hereinafter will be referred to as “B6”), and ICR strain were purchased from Crea Japan Company Limited and used.

A fertilized egg to be used was produced by fertilizing sperm of a male B6 mouse and an egg cell of a female BDF1 mouse by using a general in vitro fertilization (IVF) method.

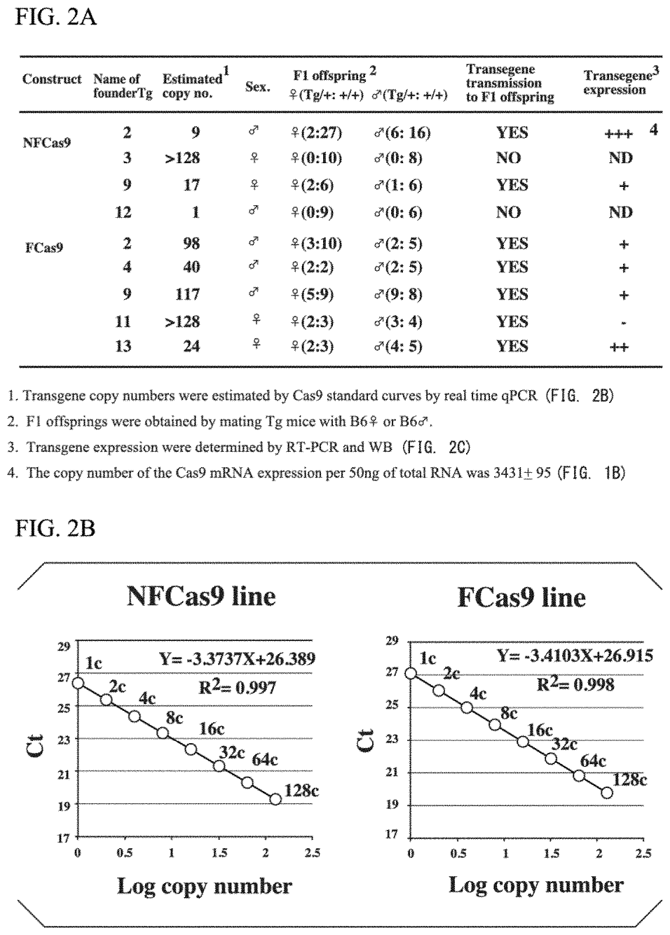

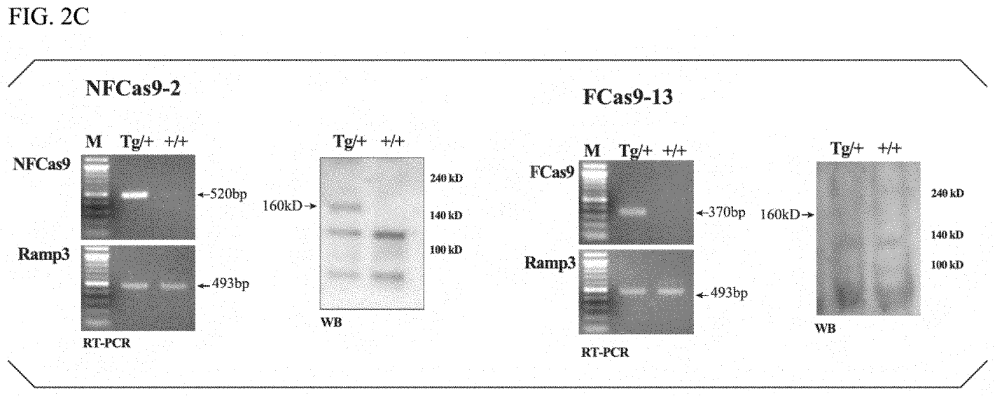

Subsequently, an injection solution was prepared so that each of the vectors containing the two types of Cas9 genes prepared in (1) became 2 ng/μL. Subsequently, the injection solution was injected into the pronuclei of the fertilized mouse eggs (56 for NFCas9 and 83 for FCas9). Subsequently, the fertilized egg into which the gene was introduced was implanted into the oviduct of an ICR pseudopregnant female mouse, and therefore F1 generation of the Cas9 transgenic mouse was obtained. Identification of introduction of Cas9 was carried out by PCR using a CAG1620-S primer and a CAS9215-A primer. As a result, 4 lines of a NFCas9 mouse and 5 lines of FCas9 were established. The characteristics of each line are shown in FIG. 2A.

Based on FIG. 2A, it was confirmed that the Cas9 transgene was transferred to the pup mouse in all 2 lines of the NFCas9 mouse and 5 lines of the FCas9 mouse.

Expression of Cas9 in fibroblasts isolated from the F1 generation of all 7 lines was confirmed by quantitative real-time PCR, RT-PCR, and western blot analysis (refer to FIG. 2C).

A method for producing a calibration curve of Cas9 (refer to FIG. 2B) for quantitative real-time PCR is as follows.

PCR was carried out using a DNA solution containing, as a template, 100 ng of genomic DNA and a dilution series of a pCAG-NFCas9 vector or a pCAG-FCas9 vector (1, 2, 4, 8, 16, 32, 64, and 128-fold copies per 100 ng genomic DNA), and a calibration curve was drawn from an amount of the obtained PCR product.

Based on FIG. 2A, the NFCas9-2 line among the established mice highly expressed Cas9 and was thus used for future analysis.

(3) Confirmation of Cas9-Introduced Locus in NFCas9-2 Line

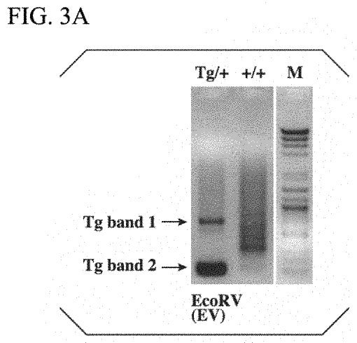

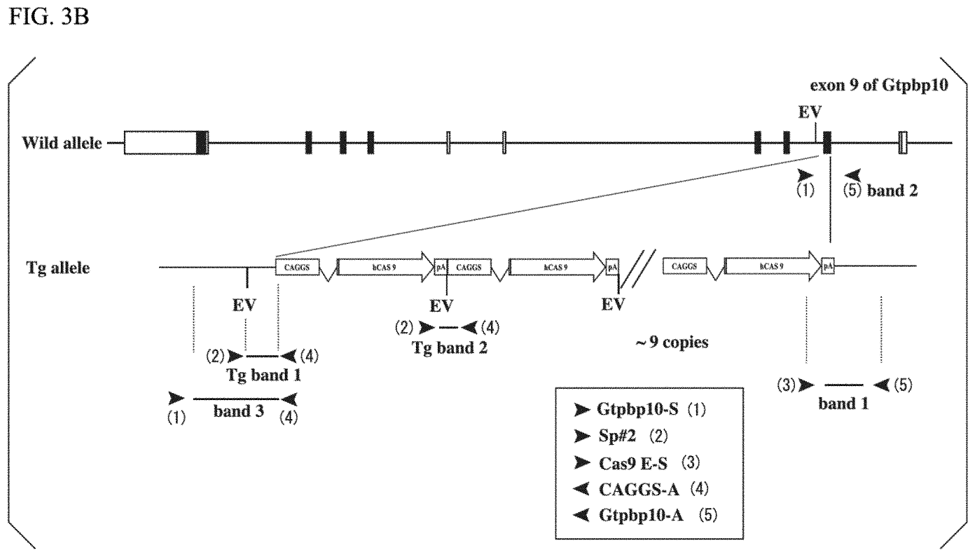

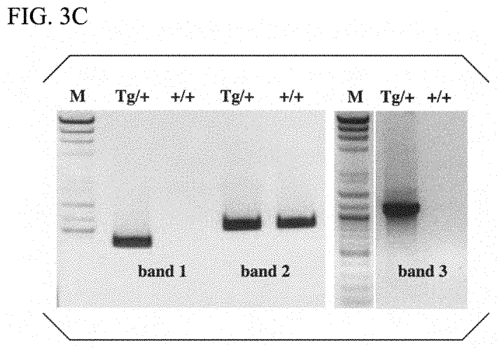

A locus into which Cas9 was introduced in the NFCas9-2 line was identified by being subjected to splinkerette PCR (spPCR) by using EcoRV splinkerette oligonucleotide (sp-EV Top primer and sp-Bottom primer) (refer to Potter, C. J. & Luo, L. Splinkerette PCR for mapping transposable elements in Drosophila. PloS One 5, e10168 (2010)). Furthermore, in order to ensure the result of spPCR, a PCR product of the genomic DNA of the NFCas9-2 line was obtained using a Gtpbp10-S primer, an Sp #2 primer, a Cas9E-S primer, a CAGGS-A primer, and a Gtpbp10-A primer. The results are shown in FIGS. 3A to 3C.

Based on FIGS. 3A to 3C, it was found that the NFCas9-2 line had nine NFCas9 transgenes in the cells, which were inserted into the Gtpbp10 gene.

### [Test Example 1] Confirmation Test on Genetic, Morphological, and Physiological Properties of NFCas9-2 Line Mouse

(1) Crossing Using NFCas9-2 Line Mouse

Male or female mice of the NFCas9-2 line were crossed with the NFCas9-2 line mouse and a wild-type BDF1 strain or a wild-type B6 strain mouse. As a result, although detailed data are not shown, a transmission rate of the transgene to the pup was in accordance with Mendel's law.

In addition, the NFCas9-2 line also grew normally in an individual into which NFCas9 was homozygously introduced, and an average number of pups was equivalent to that of a wild-type mouse of the BDF1 strain (6 pups/birth).

(2) Confirmation of Constant Expression Level of Cas9 mRNA

Cas9 mRNA in each tissue isolated from the NFCas9-2 line mouse was confimned by quantitative real-time PCR. The results are shown in FIG. 1B.

Based on FIG. 1B, it became clear that expression of Cas9 mRNA was particularly high in the heart, muscle and testis.

(3) Confirmation of Morphological Change in NFCas9-2 Line Mouse

Crossing of a female NFCas9-2 line mouse of with a wild-type BDF1 strain mouse was carried out. Subsequently, weights of the obtained littermates, a mouse of heterozygous NFCas9 (hereinafter will be referred to as “Tg/+ mouse”) and a mouse not having NFCas9 (hereinafter will be referred to as “+/+ mouse”), were each measured from 4 weeks old to 10 weeks old. The results are shown in FIG. 1C.

Based on FIG. 1C, in the Tg/+ mouse and +/+ mouse, an increase in weight tended to be the same and no difference in morphological change was recognized.

In addition, in the Tg/+ mouse and +/+ mouse, each tissue was taken out and a tissue fragment was produced. Subsequently, hematoxylin and eosin stain was carried out by a general method. The results are shown in FIG. 1D.

Based on FIG. 1D, in the heart, muscle, and testis, high expression of Cas9 mRNA was recognized in the Tg/+ mouse, and no morphological difference between +/+ mouse was recognized.

(4) Confirmation of Physiological Marker of NFCas9-2 Line Mouse

Blood was collected from 10-week-old Tg/+ mouse and +/+ mouse, and blood urea nitrogen (BUN), creatinine (Cr), aspartate aminotransferase (AST), and alanine aminotransferase (ALT), which are generally known as physiological markers in serum, were tested in SRL Corporation. The results are shown in FIG. 1E.

Various physiological markers were confirmed based on FIG. 1E.

### [Test Example 2] Ramp2 Genetic Modification Test

In the fertilized eggs derived from the Cas9 mouse, protein and mRNA of Cas9 (both or any one thereof) (hereinafter will be referred to as maCas9) were accumulated in the cell as a maternal factor, and it was considered that the Cas9 transgene is not required for genomic editing, and therefore the following test was carried out.

(1) Introduction of Guide RNA

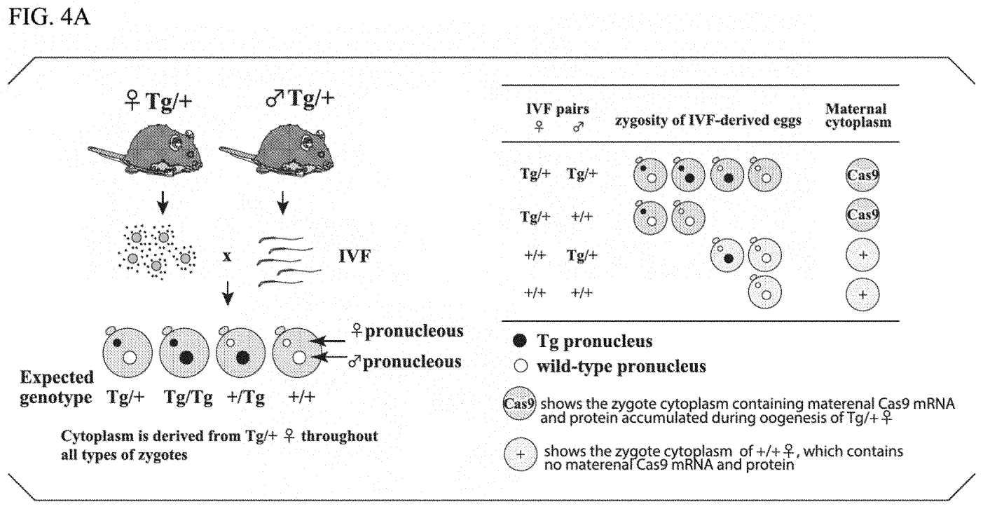

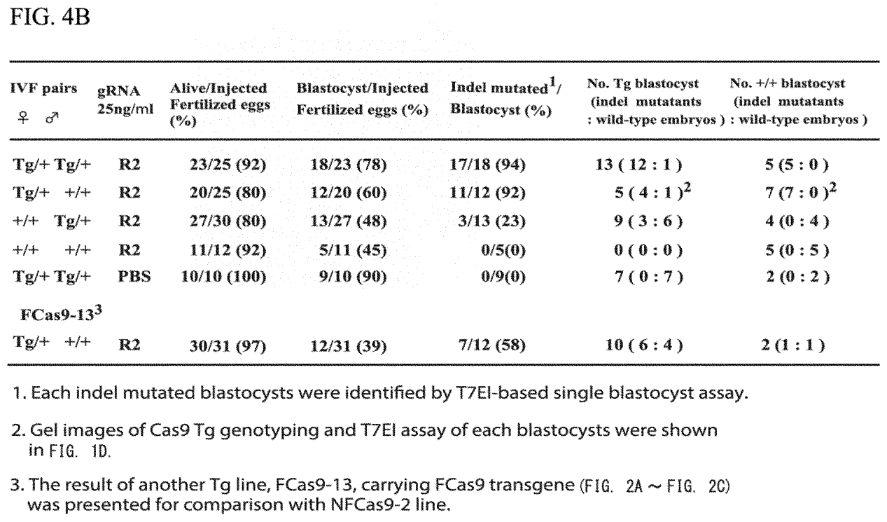

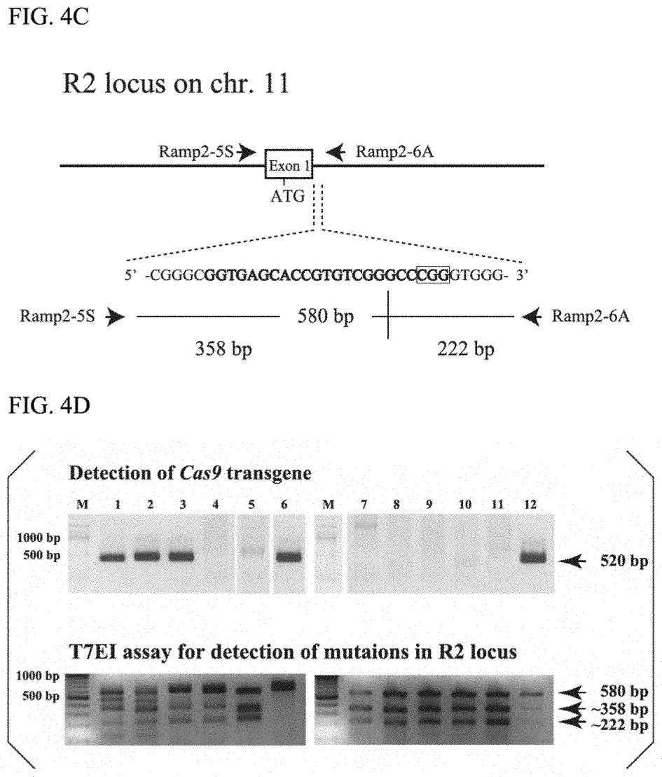

Only R2-gRNA (SEQ ID NO: 27) for Ramp2 gene modification was microinjected into the pronucleus of the fertilized eggs obtained by artificial fertilization of various crossing pairs. A genotype in the fertilized eggs produced by crossing is shown in FIG. 4A. In addition, a base sequence homologous to the Ramp2 gene in R2-gRNA is shown in FIG. 4C. In FIG. 4A, subsequently, the injected fertilized eggs were cultured to blastocysts in potassium-supplements simplex-optimized medium (KSOM).

(2) Determination of Cas9 Genotype

In regard to one embryo of the blastocysts, Cas9 genotype was determined using the same method as in (3) of Example 1. The results are shown in FIG. 4B.

(3) T7 endonuclease I (T7EI) Assay

PCR was carried out from a nuclear extract of one embryo of the blastocysts using a Ramp 2-5S primer and a Ramp 2-6A, and therefore a PCR product was obtained. The PCR product was added to T7 endonuclease I (manufactured by New England BioLabs Japan), and therefore a DNA fragment was obtained. Subsequently, a fragment cleaved by agarose electrophoresis was confirmed. The results are shown in FIG. 4D.

Based on FIGS. 4B and 4D, in a case where artificial fertilization was carried out between the Tg/+ mice, or between a Tg/+ female mouse and a +/+ male mouse, it was found that an indel mutation (a genetic mutation due to base insertion or deletion) occurred at a high rate of 92% to 94%. Accordingly, it became clear that genomic editing is possible at a high rate by Cas9 in the Tg fertilized egg.

On the other hand, in a case where artificial fertilization was carried out between a +/+ female mouse and a Tg/+ male mouse, an indel mutation was 23%, which is a low rate. Furthermore, the inventors of the present invention have investigated a relationship between a genotype of a blastocyst and a genomic editing ability, and therefore have found that an indel mutation occurs at a high rate even in a blastocyst in which the Cas9 transgene is not present.

In addition, based on FIG. 4B, in a case where in vitro fertilization was cared out between Tg/+ mice or between a Tg/+ female mouse and a +/+ male mouse, in all +/+ blastocysts, a Ramp 2 locus (5+7/5+7) had a mutation. It is known that genes on the genome are not yet expressed in cells of a fertilized egg stage. Transcripts of genes on the genome are gradually made until the 2-cell stage.

Therefore, based on this result, it is considered that protein and mRNA of maCas9 (both or any one thereof) are responsible for genomic editing in the fertilized egg.

In addition, as shown in FIG. 4B, it was possible to confirm this phenomenon in another line, FCas9-13.

### [Test Example 3] Ramp1 Gene and Ramp2 Gene Modification Test

(1) Introduction of Guide RNA

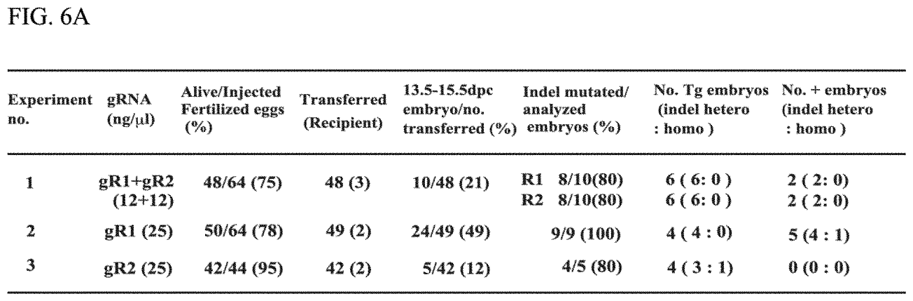

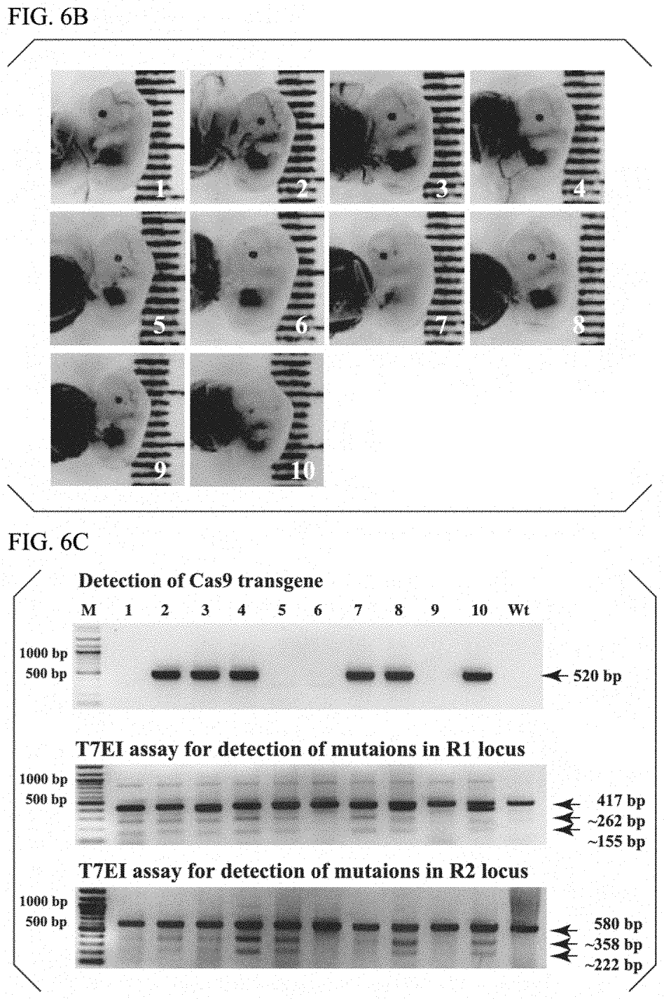

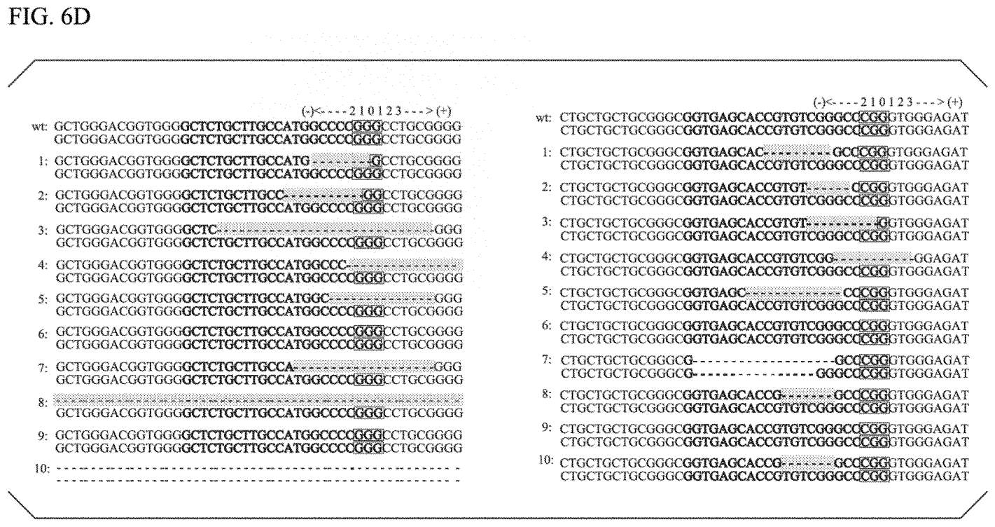

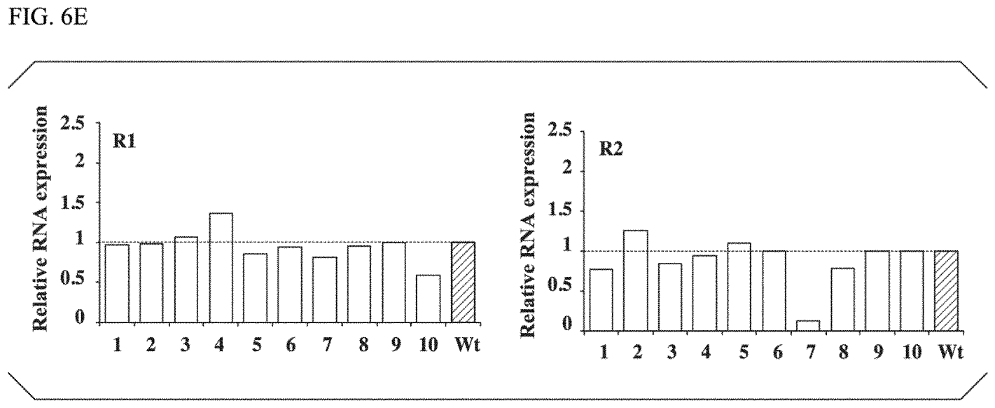

Any one of or both of R-gRNA (SEQ ID NO: 26) for Ramp1 gene modification and R2-gRNA (SEQ ID NO: 27) for Ramp2 gene modification were microinjected into the pronucleus of the fertilized eggs obtained by artificial fertilization of a Tg/+ female mouse and a +/+ male mouse. Subsequently, the injected fertilized eggs were cultured to blastocysts in potassium-supplements simplex-optimized medium (KSOM). Some of the blastocysts into which R1-gRNA and R2-gRNA were introduced at the same time were implanted into the uterus of an ICR strain pseudopregnant female mouse, 13.5 to 15.5 days after implantation, embryos were obtained. The form of embryos of 10 pups 13.5 days after the implantation is shown in FIG. 6B.

(2) Determination of Presence or Absence of Mutation

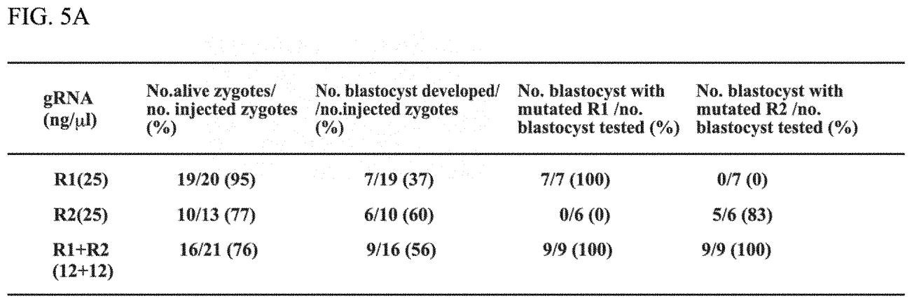

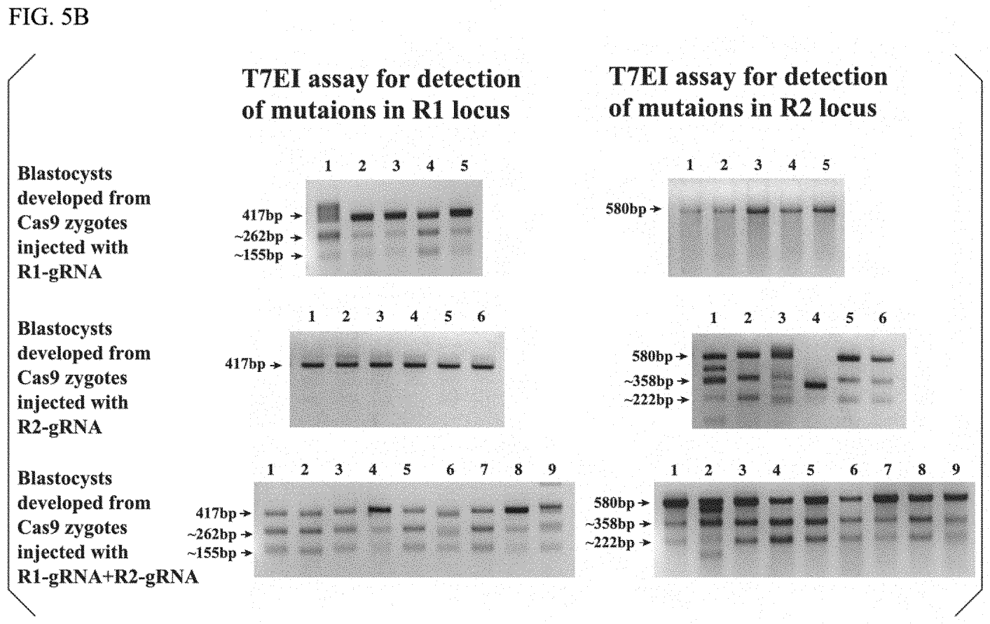

A nuclear extract of one embryo of the blastocysts or a DNA solution prepared from the tail or the hand of the embryos 13.5 days after the implantation was subjected to PCR using a Ramp1-2S primer and a Ramp1-2A primer, and a Ramp2-5S primer and a Ramp 2-6A, and therefore a PCR product was obtained. Subsequently, the PCR product was sequenced using BigDye Terminator v.3.1 and ABI Genetic Analyzer 3130 Ramp1. The presence or absence of mutation of Ramp1 gene and Ramp2 gene was determined. The results are shown in FIG. 5A (blastocysts), and in FIGS. 6A and 6D (embryos 13.5 days after the implantation).

(3) T7EI Assay

The nuclear extract of one embryo of the blastocysts or the DNA solution prepared from the tail or the hand of the embryos 13.5 days after the implantation was subjected to T7EI Assay using the same method as in (3) of Test Example 2. For blastocysts into which R1-gRNA was introduced, a Ramp1-2S primer and a Ramp1-2A primer were used. The results are shown in FIG. 5B (blastocysts) or FIG. 6C (embryos 13.5 days after the implantation).

Based on FIGS. 5A and 5B, in a case where only R1-gRNA was injected into the fertilized egg, the indel mutation was recognized only at the Ramp1 locus. The same results were also confirmed in a case where only R2-gRNA was injected into the fertilized egg. In a case where R1-gRNA and R2-gRNA were injected at the same time, simultaneous mutagenesis was observed at both loci.

Based on the above results, it could be confirmed by the blastocyst that genomic editing in the fertilized egg injected only with the guide RNA works specifically at the target locus.

Furthermore, when the indel mutation in the stage of embryos derived from the fertilized egg (Tg/+ female mouse and +/+ male mouse) injected with R1-gRNA and R2-gRNA at the same time was examined, almost all of the embryos were hetero- or homo-genetically modified embryos (refer to FIG. 6A).

In addition, the presence or absence of mutation at each of the three types of off-target sites predicted in each of the Ramp1 gene and Ramp2 gene was examined by subjecting a DNA solution prepared from the tail or the hand of the obtained embryos of ten pups (using Experiment 1 shown in FIG. 6A) to PCR using a primer for off-target analysis shown in the above table. As a result, not one mutation was recognized.

### [Test Example 4] Comparative Examination 1 with Method of the Related Art

Subsequently, a genomic editing method for microinjecting synthetic Cas9 mRNA and guide RNA into a fertilized egg, and a genomic editing method by maCas9 of the present invention were compared.

(1) Introduction of Guide RNA

Fertilized eggs containing maCas9 were produced by artificial fertilization using Tg/+ egg cell and +/+ sperm. Subsequently, only R2-gRNA (25 ng/μL) was microinjected into the cytoplasm and the pronucleus of the fertilized eggs containing maCas9 (genotype: Tg/+ or +/+). As a control group, both synthetic Cas9 mRNA (50 ng/μL) and R2-gRNA (25 ng/μL) were injected into the cytoplasm and the pronucleus of the wild-type fertilized eggs (not containing maCas9; genotype +/+). These fertilized eggs were cultured to blastocysts.

(2) Determination of Genotype and Presence or Absence of Mutation

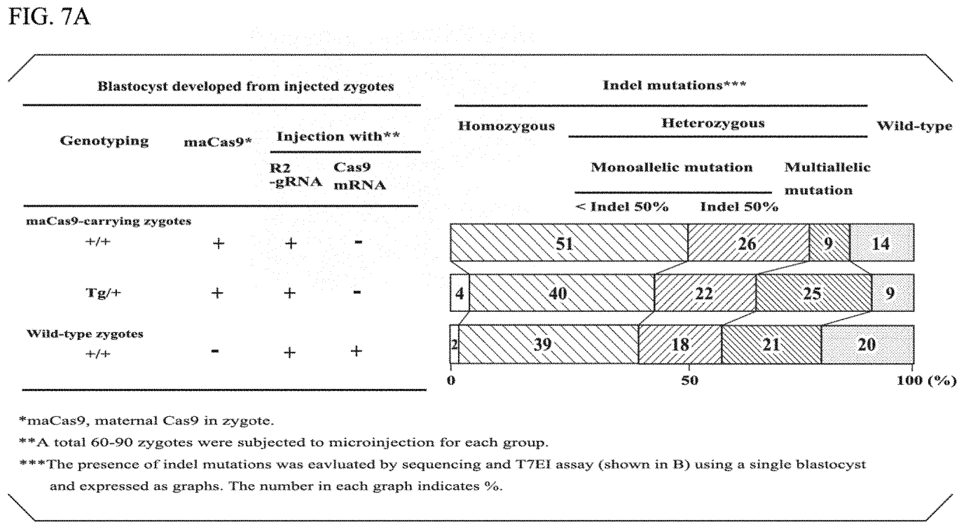

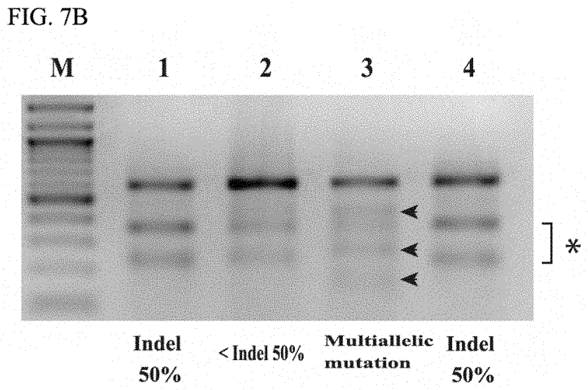

A genotype was determined by using the same method as in (3) of Example 1. Furthermore, the presence or absence of mutation was determined by using the same method as in (2) of Test Example 3. The results are shown in FIG. 7A.

(3) T7EI Assay

T7EI assay was carried out on the blastocysts having a heterozygous mutant (monoallelic or multiallelic) using the same method as in (3) of Test Example 2. The results are shown in FIG. 7B.

In FIG. 7B, “<Indel 50%” in lane 2 indicates that the total intensity of two cleaved bands is smaller than that of a parent band. In addition, in “Indel 50%” in lanes 1 and 4, the total intensity of two cleaved bands is almost the same as a parent band. In “Multiallelic mutation” in lane 3, two or more cleaved bands are generated.

Based on FIG. 7A, induction rates of the mutation at the target site and the indel mutant types (homozygous (biallelic), monoallelic, or multiallelic KO) were statistically equivalent to three experimental groups (maCas9-carrying +/+ and Tg/+ fertilized eggs, and a control group (maCas9-non-carrying +/+ fertilized egg)) (p>0.05, oneway-ANOVA). In addition, nonmosaic variants (50% indel mutation) and mosaic variants (<50% monoallelic indel mutation and multiallelic mutation) in the maCas9-carrying +/+ and Tg/+ fertilized eggs were also statistically equivalent to those in the control group (p>0.05, oneway-ANOVA). Furthermore, it was recognized that an induction rate of the multiallelic mosaic variants in the maCas9-carrying +/+ fertilized eggs tended to be lower compared to that of the maCas9-carrying Tg/+ fertilized eggs and the control group, but an induction rate of the nonmosaic variants thereof (50% indel mutation) tended to be higher.

In addition, the presence or absence of base sequence mutation at the three types of the predicted off-target sites in blastocysts generated from maCas9-carrying fertilized eggs injected with R2-gRNA was examined by PCR using a primer for off-target analysis shown in the above table. As a result, there was no mutation even at one location.

Based on the above result, the genomic editing ability by maCas9 is considered to be equivalent to that in the method for injecting synthetic Cas9 into fertilized eggs. In addition, it became clear that in the genomic editing by maCas9, the mosaic variants significantly reduced compared with the method for injecting synthetic Cas9 into fertilized eggs.

### [Test Example 5] Confirmation Test of Simultaneous Generation of Multiple Gene Mutations

Subsequently, the number of mutations which can be induced simultaneously in embryos in which maCas9 is not present in the Cas9 transgene was examined.

(1) Introduction of Guide RNA

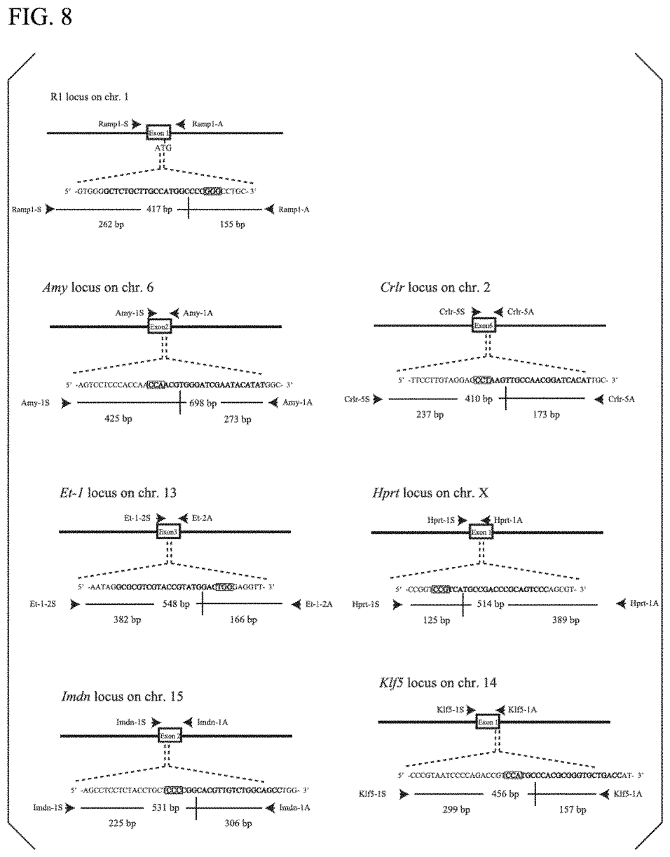

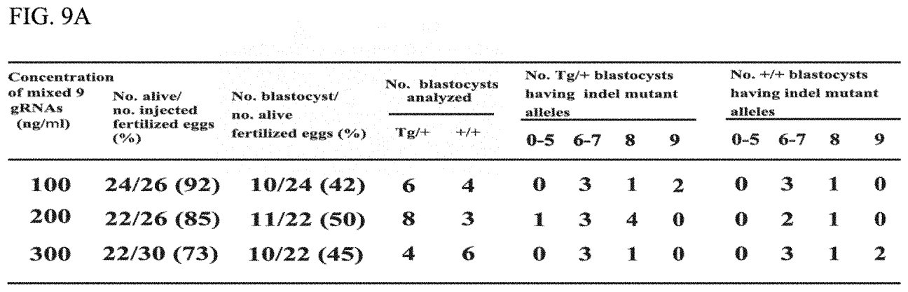

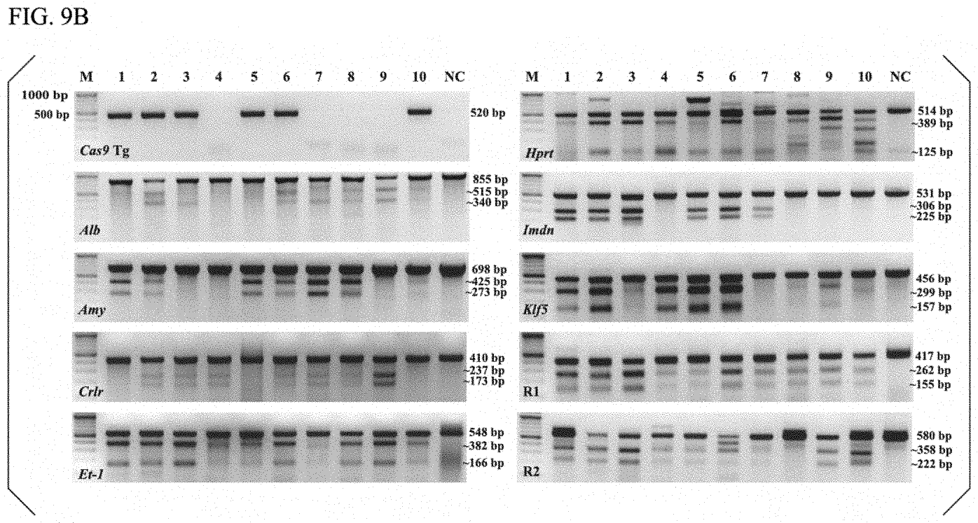

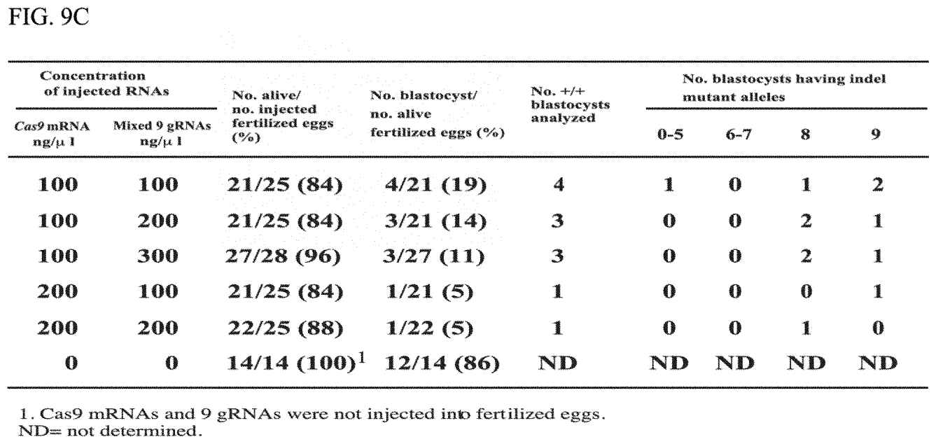

Nine different types of guide RNAs were microinjected into fertilized eggs prepared by in vitro fertilization of a Tg/+ female mouse and a +/+ male mouse. The target genes are nine types, which are Ramp1 gene, Ramp2 gene, Amy gene, Crlr gene, Et-1 gene, Hprt gene, Imdn gene, and Klf5 gene. The sequence of the guide RNA is as shown in “Guide RNA” in the above table, and FIGS. 4C and 8. As a control group, these nine types of the guide RNAs and synthetic Cas9 mRNA (refer to FIG. 9C) were microinjected into fertilized eggs prepared by in vitro fertilization of a +/+ female mouse and a +/+ male mouse.

(2) Determination of Genotype and Presence or Absence of Mutation

A genotype was determined by using the same method as in (3) of Example 1. Furthermore, the presence or absence of mutation was determined by using the same method as in (2) of Test Example 3. As a primer at this time, the Amy-1S primer and the Amy-1A primer, the Crlr-5S primer and the Crlr-5A primer, the Et-1-1S primer and the Et-1-1A primer, the Hprt-1S primer and the Hprt-1A primer, the Imdn-1S primer and the Imdn-1A primer, the Klf5-1S primer and the Klf5-1A primer, the Ramp1-2S primer and the Ramp1-2A, and the Ramp2-5S primer and the Ramp 2-6A, which are described in the above tables <Primer>, were used. The results are shown in FIG. 9A.

(3) T7EI Assay

T7EI Assay was carried out using the same method as in (3) of Test Example 2. The results are shown in FIG. 9B. A primer used is the same as that exemplified in (2) of the present test example.

Based on FIGS. 9A and 9C, it could be confirmed that in the blastocysts using the method of the present invention, indel mutations were simultaneously generated at an average of 7.3 loci, which was equivalent to that of the control group. In addition, two cases of each of nine types of simultaneous indel mutations were observed in the blastocysts derived from the maCas9-carrying +/+ and Tg/+ fertilized eggs.

Furthermore, importantly, a development rate of the blastocyst using the method of the present invention was 42% to 50%, but a development rate of the blastocyst of the control group was 5% to 19%, which was extremely low to obtain the pup.

Based on the above result, it can be said that the fertilized eggs containing maCas9 can be simultaneously introduced with mutations into multilocus and thus are extremely useful for the production of a genetic modification mouse having multigene.

### [Test Example 6] In Vitro Genetic Modification Test

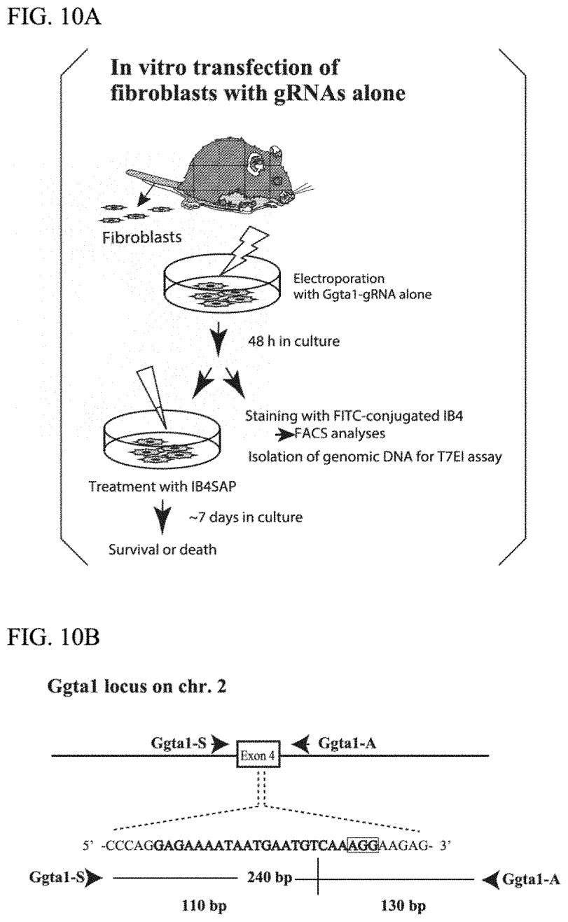

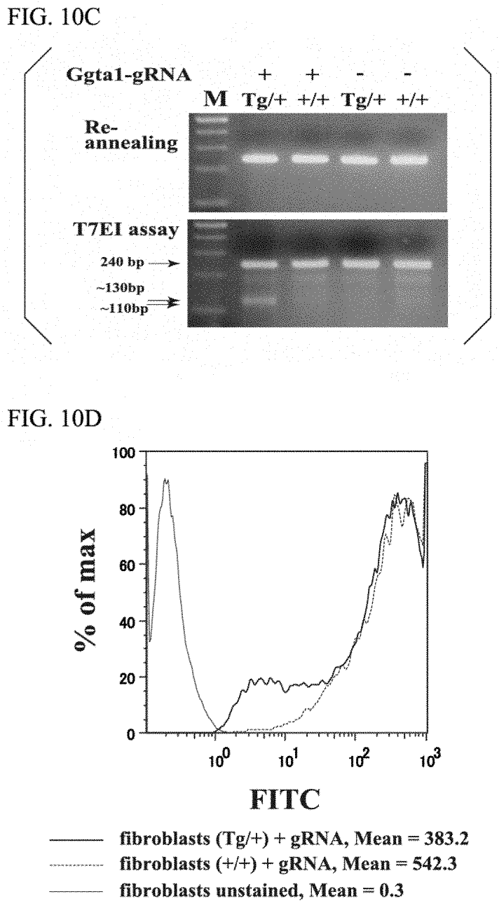

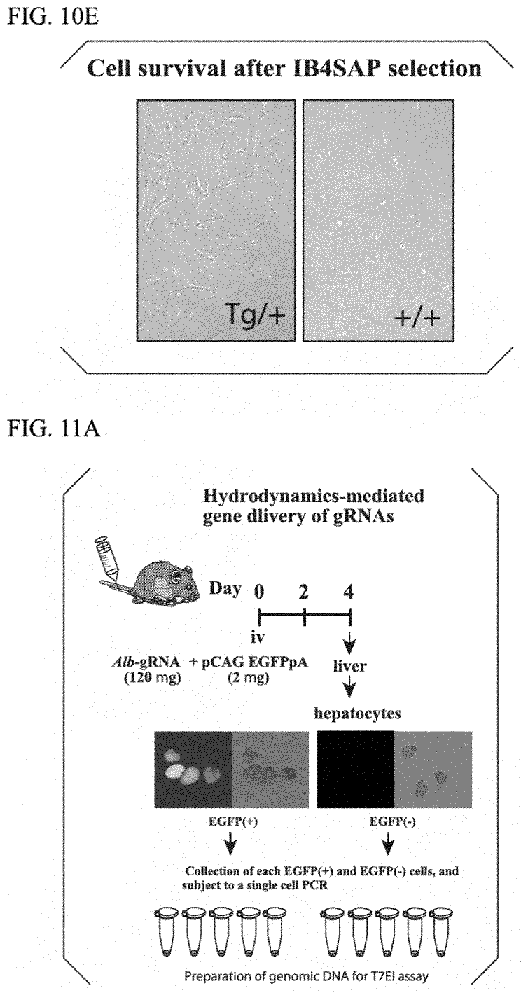

Next, in order to verify the practicality of the Cas9 mouse in in vitro genetic modification, disruption of Ggat1 gene involved in synthesis of α-Gal epitope of the cell surface was investigated. FIG. 10A shows a protocol in the present test.

(1) Introduction of Guide RNA

Fibroblasts were primarily cultured from the tails of a Tg/+ and +/+ pup of the Cas9 mouse. The culturing was carried out for 5 days using DMEM (Dulbecco's modified Eagle's essential medium) containing 10% fetal bovine serum. Subsequently, 100 μg/mL of Ggta1 gRNA (SEQ ID NO: 22) dissolved in phosphate-buffered saline (calcium and magnesium-free, pH 7.4) was transiently introduced into 5×10⁶ cultured fibroblasts by an electroporation method using Gene-Pulser II (Manufactured by Bio-Rad Laboratories, Inc.). Abase sequence homologous to the Ggat1 gene in Ggta1 gRNA is shown in FIG. 10B.

(2) T7EI Assay

A PCR product was prepared by PCR from a homogenate of the fibroblasts after introduction of the guide RNA by using the Ggta1-S primer and the Ggta1-A primer. Subsequently, T7EI Assay was carried out using the same method as in (3) of Test Example 2. The results are shown in FIG. 10C.

Based on FIG. 10C, it was confirmed that the indel mutation occurred only in the Ggta1 gene.

(3) FACS Analysis

By using the fibroblasts after introduction of the guide RNA, FACS analysis was carried out by utilizing the fact that FITC-labeled isolectin, BS-I-B4 (IB4) specifically recognizes α-Gal epitope. The results are shown in FIG. 10D.

Based on FIG. 10D, a decrease in α-Gal epitope expression (up to 30% reduction compared to a wild-type cell), which was the target, was confirmed.

(4) Survival Assay

The fibroblasts after introduction of the guide RNA were treated with IB4-saporin conjugate (IB4SAP). The death of cells having 2 genes of Ggta1 (+/+ type) and cells having 1 gene knocked out of Ggta1 (KO/+ type) was recognized. On the other hand, survival of cells considered to be biallelic Ggta1 KO was observed (refer to FIG. 10E). The survival rate was 20 (colony) per 5×10⁵ cells, and an induction efficiency of gene defect estimated from the above rate was estimated to be 0.004%.

Based on the above result, it was confirmed that the primary cultured cells prepared from the Cas9 mouse line caused genomic editing by CRISPR/Cas9 only by introducing the guide RNA.

### [Test Example 7] In Vivo Genetic Modification

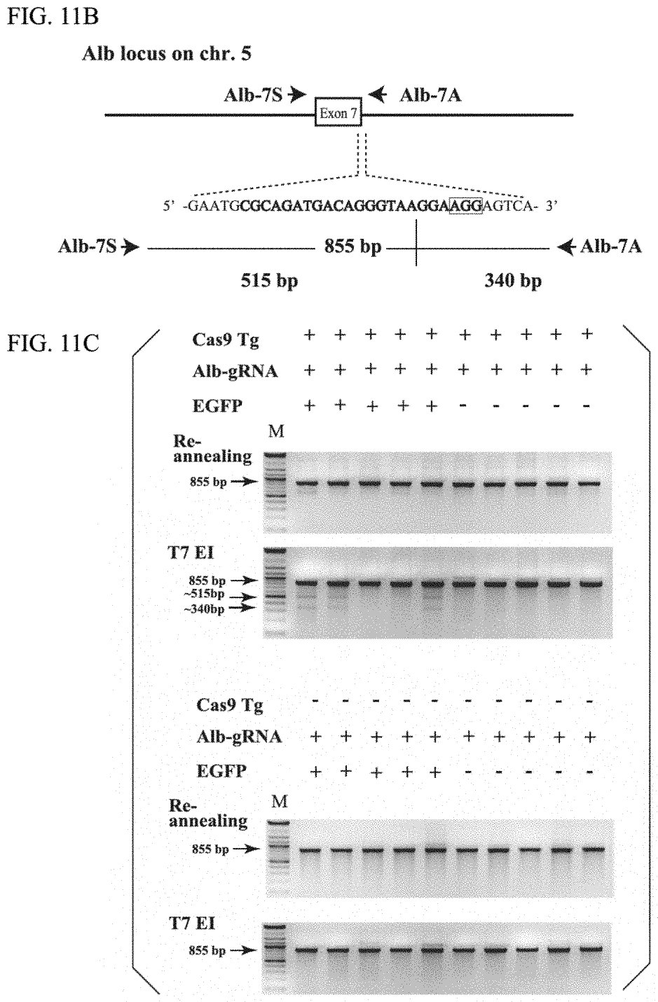

To verify that in vivo genomic editing is possible by administration of the guide RNA in the Cas9 mouse, disruption of genes of hepatocytes by a hydrodynamic guide RNA introduction method was investigated. FIG. 11A shows a protocol in the present test.

(1) Introduction of Guide RNA

120 μg of albumin (Alb) gRNA (SEQ ID NO: 18) and 4 μg of pCAG-EGFPpA were injected simultaneously into the tail vein of the Cas9 mouse and the wild-type mouse by using TransIT-QR Hydrodynamic Delivery Solution kit (manufactured by Mirus Bio LLC). A base sequence homologous to Alb1 gene in Alb gRNA is shown in FIG. 11B.

Thereafter, the hepatocytes were isolated from each mouse. By this method, EGFP-positive cells were obtained at a ratio of 0.1% (1/1000 cells).

(2) T7EI Assay

30 EGFP-positive cells and 30 EGFP-negative cells were isolated from the hepatocytes derived from the Cas9 mouse. Similarly, 30 EGFP-positive cells and 30 EGFP-negative cells were isolated from the hepatocytes derived from the wild-type mouse. Subsequently, a PCR product was prepared by PCR from a homogenate of each cell by using the Alb-7S primer and the Alb-7A primer. Subsequently, T7EI Assay was carried out using the same method as in (3) of Test Example 2 so as to examine the presence or absence of indel mutation. The results are shown in FIG. 11C.

Based on FIG. 11C, the indel mutation of Alb1 gene was recognized only in the hepatocytes derived from the EFGP-positive-Cas9 mouse at a rate of about 50% (15/30), and a ratio of the hepatocytes in which the mutation occurred was calculated to be 0.05% (1/2000 cells). In the hepatocytes derived from the EGFP-negative-Cas9 mouse, the hepatocytes derived from the EGFP-positive-wild type mouse, and the hepatocytes derived from the EGFP-negative-wild type mouse, the indel mutation of Alb1 gene was not recognized.

Based on the above results, it became clear that in vivo administration of the guide RNA causes the indel mutation of the endogenous target gene of the Cas9 mouse.

### [Test Example 8] Comparative Examination 2 with Method of the Related Art

Based on Test Example 4, it was shown that the genomic editing ability by maCas9 was equivalent to that in the method for injecting synthetic Cas9 mRNA into fertilized eggs by the injection of Ramp2 gRNA into the fertilized egg and its analysis at the blastocyst stage, and it was shown that an induction rate of the multiallelic mosaic variants in the maCas9-carrying +/+ fertilized eggs tended to be lower compared to that of the maCas9-carrying Tg/+ fertilized eggs and control group. In order to more strongly back up this finding, ET-1-gRNA as gRNA of another gene was introduced into fertilized eggs, and these fertilized eggs were developed up to embryos of 12.5 to 13 days old, and an indel mutation ratio and a mosaic ratio were examined.

(1) Introduction of Guide RNA

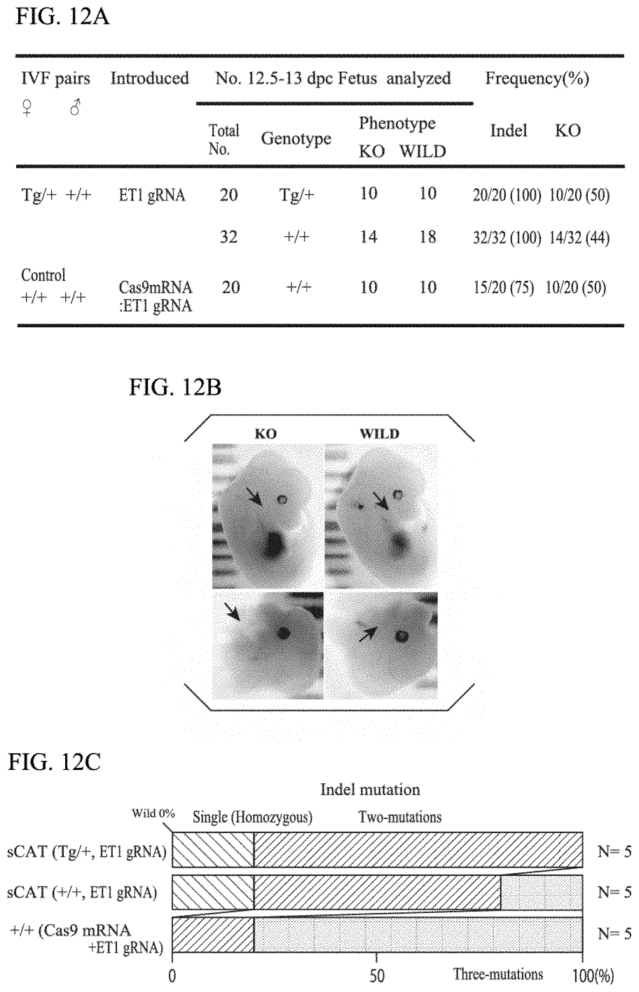

Fertilized eggs containing maCas9 were produced by artificial fertilization using Tg/+ egg cell and +/+ sperm. Subsequently, only ET-1-gRNA (200 ng/μL) was introduced into the cytoplasm and the pronucleus of the fertilized eggs containing maCas9 (genotype: Tg/+ or +/+) by an electroporation method. As a control group, both synthetic Cas9 mRNA (100 ng/μL) and ET-1-gRNA (200 ng/μL) were introduced into the wild-type fertilized eggs (not containing maCas9; genotype +/+) by using the electroporation method. These fertilized eggs were cultured to blastocysts. Each of the blastocysts into which ET-1-gRNA was introduced was implanted into the uterus of an ICR strain pseudopregnant female mouse, 12.5 to 13 days after implantation, embryos were obtained. The form of embryos of ten pups 12.5 to 13 days after the implantation is shown in FIG. 12B.

(2) Determination of Genotype and Presence or Absence of Mutation

A genotype was determined by using the same method as in (3) of Example 1. Furthermore, the presence or absence of mutation was determined by using the same method as in (2) of Test Example 3 except that the Et-1-1S primer and the Et-1-1A primer were used. Furthermore, fetuses of the Et-1 KO phenotype (each N=5) among Tg/+ and +/+, and the control group were randomly selected to be analyzed, and therefore a mosaic ratio was calculated. The results are shown in FIG. 12C.

Based on FIG. 12A, with respect to 75% in the control group, the indel mutation was 100% in the Tg/+ and +/+ fetuses of the Cas9 mouse, which was the high ratio.

In addition, based on FIG. 12B, abnormality of jaw formation was observed in the Et-1 knockout (KO) phenotype.

Based on FIG. 12A, with respect to 50% in the control group, this Et-1 KO phenotype was 50% and 44% in each of the Tg/+ and +/+ fetuses of the Cas9 mouse.

In addition, based on FIG. 12C, in regard to a mosaic ratio, there were mostly two types of the indel mutations in the Tg/+ and +/+ fetuses of the Cas9 mouse, but one type (homozygous: only the same mutant genome) was obtained. On the other hand, in the control group, there were mostly three types.

Based on the above results, it was shown that the genomic editing ability by maCas9 was equivalent to that in the method for injecting synthetic Cas9 mRNA into fertilized eggs, but a mosaic ratio was lower compared to that of the method for injecting Cas9 mRNA/gRNA of the related art, and accordingly, characteristics of the genomic editing ability by maCas9 (high efficiency and low mosaic ratio) were again verified in the Et-1 gene.

### [Test Example 9] Test of Examination on gRNA Introduction Timing

The influence on the indel ratio and the mosaic ratio by a difference in timing of gRNA introduction into the fertilized egg derived from Cas9 mouse was examined.

(1) Introduction of Guide RNA

Fertilized eggs containing maCas9 were produced by artificial fertilization using Tg/+ egg cell and +/+ sperm. Subsequently, only ET-1-gRNA (200 ng/μL) was introduced into the cytoplasm and the pronucleus of the fertilized eggs containing maCas9 (genotype: Tg/+ or +/+) after 6 to 8 hours, or 10 to 12 hours from insemination, by an electroporation method. These fertilized eggs were cultured to blastocysts.

(2) Determination of Genotype and Presence or Absence of Mutation

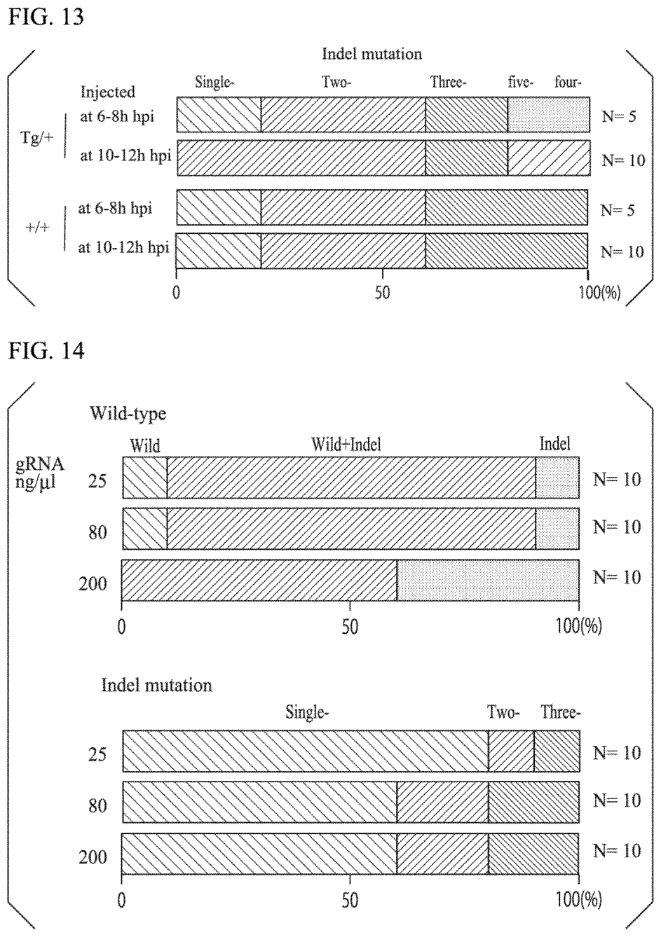

A genotype was determined by using the same method as in (3) of Example 1. Furthermore, the presence or absence of mutation was determined by using the same method as in (2) of Test Example 3 except that the Et-1-1S primer and the Et-1-1A primer were used. Furthermore, fetuses of the Et-1 KO phenotype (each N=5) among Tg/+ and +/+ were randomly selected to be analyzed, and therefore a mosaic ratio was calculated. The results are shown in FIG. 13.

Based on FIG. 13, in any of the fertilized eggs into which gRNA was introduced after 6 to 8 hours, and 10 to 12 hours from insemination, the indel ratio was high (100% in any egg).

In the Tg/+ blastocysts, in comparison under the conditions after 6 to 8 hours, and 10 to 12 hours from insemination, a ratio of a single indel genomic mutation decreased under the condition after 10 to 12 hours, and an increase of multiallelic mosaic mutation was recognized.

On the other hand, in the +/+ blastocysts under the conditions after 6 to 8 hours, and 10 to 12 hours from insemination, no change was recognized in ratios of a single indel genomic mutations and multiallelic mosaic mutation. This was presumed to be due to the fact that gRNA introduced in the later stage exists longer without degradation than the gRNA introduced in the early stage. Furthermore, from the fact that the genotype is Tg/+ embryo, a possibility that Cas9 derived from zygote was transcribed and translated to work was considered.

Based on the above result, a change in the number of indel genomic mutation due to the timing of gRNA introduction into the Tg/+ fertilized egg was recognized. In order to reduce the multiallelic mosaic mutation, it was suggested that it is preferable to introduce gRNA into the fertilized egg at the early stage. On the other hand, it was suggested that for the purpose of obtaining an individual having many multiallelic mosaic mutations, the fertilized egg of the late stage is preferably utilized.

### [Test Example 10] Test of Examination on gRNA Concentration

The influence on the indel mutation ability by a change in concentration of introduced gRNA was examined.

(1) Introduction of Guide RNA

Fertilized eggs containing maCas9 were produced by artificial fertilization using Tg/+ egg cell and +/+ sperm. Subsequently, at three different concentrations of 25 ng/μL, 80 ng/μL, and 200 ng/μL, only ET-1-gRNA was introduced into the cytoplasm and the pronucleus of the fertilized eggs containing maCas9 (genotype: Tg/+ or +/+) 6 to 8 hours from insemination by using an electroporation method. These fertilized eggs were cultured to blastocysts.

(2) Determination of Genotype and Presence or Absence of Mutation

A genotype was determined by using the same method as in (3) of Example 1. Furthermore, the presence or absence of mutation was determined by using the same method as in (2) of Test Example 3 except that the Et-1-1S primer and the Et-1-1A primer were used. Furthermore, fetuses of the Et-1 KO phenotype (each N=5) among Tg/+ and +/+ were randomly selected to be analyzed, and therefore a mosaic ratio was calculated. The results are shown in FIG. 14.

Based on FIG. 14, the indel mutation was 90% to 100%, which is a high ratio, under the conditions at any concentration of 25 ng/μL, 80 ng/μL, and 200 ng/μL. The non-indel mutation (wild) was not recognized under the condition of 200 ng/μL ET-1-gRNA introduction, and the embryo of only the indel mutant genome reached up to 40%.

In addition, it was recognized that the number of the indel mutation per embryo increased concentration-dependently. That is, under the condition of 25 ng/μL ET-1-gRNA introduction, 80% was one type of the indel mutation (including homozygous (only the same mutant genome) and heterozygous (only a wild-type and one type of indel mutant genomes)). On the other hand, under the conditions of 80 ng/μL and 200 ng/μL ET-1-gRNA introductions, the indel mutation ratio decreased down to 60% and 20%, respectively.

In addition, an increase of two types of the indel mutations (including only two types of the indel mutant genomes, and a wild-type and two types of indel mutant genomes) was recognized.

Based on the above results, it became clear that it is possible to change the number of embryo indel mutant genomes by changing a concentration of gRNA to be introduced.

In the condition at a low concentration (25 ng/μL), most were embryos including a wild-type and one type of indel mutant genomes. These are considered to be advantageous characteristics in a case where homozygosity is generated, in a case where it is desired to obtain a genetic modification individual by avoiding lethal mutation, and in a case where reversely, a heterozygous mutant individual is preferentially desired.

### [Test Example 11] Test of Knockin of Foreign Gene to Klf5 Gene

Whether knockin of a foreign gene is also possible in the fertilized egg derived from the Cas9 mouse was verified by a test of knockin of a foreign gene to Klf5 gene.

(1) Design of Guide RNA and Foreign Gene (KI Fragment)

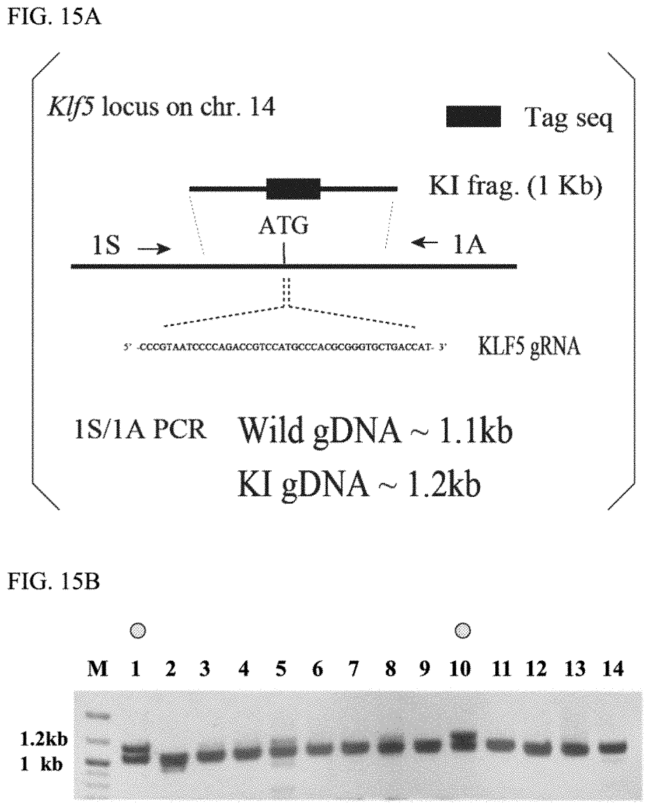

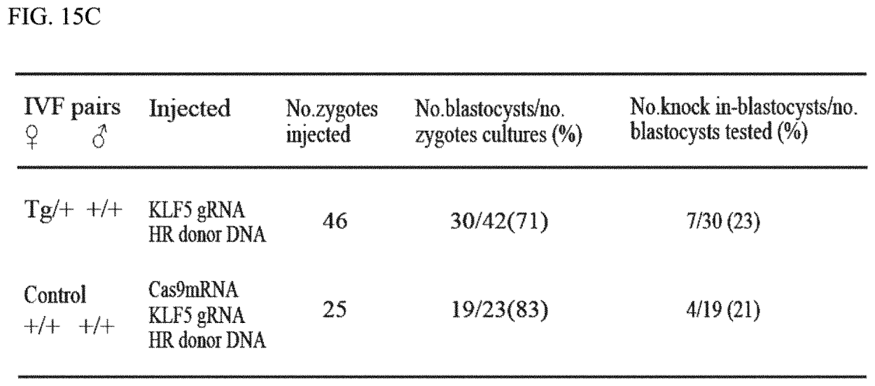

As shown in FIG. 15A, gRNA was designed near the atg base for the translation initiation of the Klf5 gene (Klf5 gRNA2: SEQ ID NO: 79). Furthermore, to the region thereof in the 5′ and 3′ directions, about 100 bp of foreign genes (a total of about 1 kb) containing a Flag-tagged sequence in which about 500 bp of homologous DNA sequences were linked were synthesized as a knockin sequence (KI fragment) (KI Fragment: SEQ ID NO: 80).

(2) Introduction of Guide RNA

Fertilized eggs containing maCas9 were produced by artificial fertilization using Tg/+ egg cell and +/+ sperm. In addition, fertilized eggs of a control group were produced by artificial fertilization using +/+ egg cell and +/+ sperm. Subsequently, Klf5 gRNA2 (10 ng/μL) produced in (1) and the KI fragment (3.3 ng/μL) produced in (1) were injected into the cytoplasm and the pronucleus of the fertilized eggs containing maCas9 (genotype: Tg/+ or +/+) 10 to 12 hours after insemination. In addition, KIf5 gRNA2 (10 ng/μL) produced in (1) and the KI fragment (3.3 ng/μL) produced in (1), and Cas9 mRNA (33 ng/μL) were injected into the cytoplasm and the pronucleus of the fertilized eggs of the control group of the same stage. These fertilized eggs were cultured to blastocysts.

(2) Determination of Genotype and Presence or Absence of Mutation

A genotype was determined by using the same method as in (3) of Example 1. In addition, a solution for PCR was prepared from each blastocyst and PCR was carried out by using PCR primers (Klf5-1S primer (SEQ ID NO: 52) and Klf5-1A primer (SEQ ID NO: 53)) set in a genomic DNA sequence outside of a homologous sequence of the KI fragment. The knockin-Klf5 gene was determined from the presence or absence of a PCR product (about 1.2 kb) having a size larger by about 0.1 kb than a wild-type PCR product (about 1.1 kb). The results are shown in FIG. 15B and FIG. 15C.

Based on FIG. 15B, the PCR product larger than the wild-type PCR product of about 1.2 kb was detected in Samples No. 1 and No. 10.

In addition, based on FIG. 15C, 7 embryos among 30 embryos of blastocysts derived from the fertilized eggs of the Cas9 mouse were knockin-embryos (23%). On the other hand, 4 embryos among 19 embryos of blastocysts derived from the fertilized eggs of the control group were knockin-embryos (21%).

Based on the above results, it was possible to knockin the foreign genes by using the fertilized eggs containing maCas9, and it became clear that the efficiency thereof was equivalent to that in the method for injecting Cas9 mRNA/gRNA of the related art.

### [Example 2] Establishment of Mouse Line Expressing Nickase Cas9 (DIOA) in its Entirety

In addition to the characteristics of the Cas9 mouse described above, establishment of a Nickase Cas9 (D10A) mouse, which is a transgenic mouse which has both a processing ability for reducing an off-target rate and a processing ability of Nick genome, and in which NickaseCas9 is expressed in its entirety, was carried out.

(1) Design of Construct for Inserting Cas9 Gene

Using, as a template, pCAG-NFCas9 pA and pCAG-FCas9 pA constructed in Example 1, PCR was carried out by using A NFL (D10A) S primer (SEQ ID NO: 81) and a NFL (D10A) A primer (SEQ ID NO: 82) so as to construct a Cas9 (D10A) vector in which the 10^{th} amino acid of Cas9 was substituted with A from D.

(2) Injection into Fertilized Mouse Egg

A fertilized egg to be used was produced by fertilizing sperm of a male B6 mouse and an egg cell of a female BDF1 mouse by using a general in vitro fertilization (IVF) method.

Subsequently, an injection solution was prepared so that each of the two types of Cas9 (D10A) vectors prepared in (1) became 2 ng/μL. Subsequently, the injection solution was injected into the pronuclei of the fertilized mouse eggs. Subsequently, the fertilized egg into which the gene was introduced was implanted into the oviduct of an ICR pseudopregnant female mouse, and therefore F1 generation of the Nickase Cas9 (D10A) transgenic mouse was obtained.

(3) Determination of Genotype

A genotype of 30 grown pups of the F1 generation was determined by using the same method as in (3) of Example 1. Identification of introduction of Cas9 (D10A) was carried out by PCR using a CAG1620 primer (SEQ ID NO: 32) and a Cas9 215-A primer (SEQ ID NO: 34). As a result, 6 pups were Tg individuals. In regard to the above 6 pups, no influence on development and survival was recognized at the present time, as in the case of the Cas9 mouse.

In addition, by continuing crossing among the above 6 pups, a Nickase Cas9 (D10A) mouse of 2 lines was established. When cDNA was prepared from major organs such as the lung and ovary of the pup of the Nickase Cas9 (D10A) mouse of 2 lines so as to examine the Cas9 (D10A) expression by Real Time PCR using the cDNA, the Cas9 (D10A) expression was confirmed.

### INDUSTRIAL APPLICABILITY

According to the method for modifying a target gene of the present invention, it is possible to edit a plurality of different genes or a plurality of locations in the same gene at the same time with high efficiency. In addition, according to the method for modifying a target gene of the present invention, a genetic modification non-human organism can be rapidly produced at a low cost. Furthermore, the method for modifying a target gene of the present invention can be utilized in studies of lifestyle diseases such as cancer or heart disease in which abnormality of many genes is involved, or disease-related genes identified by genome-wide association study (GWAS).

1. A method for modifying at least one target gene, the method comprising:
introducing a guide RNA into at least one cell obtained from an unfertilized egg cell,
wherein the unfertilized egg cell is obtained from a genetically modified non-human organism comprising a nuclear genome into which at least 3 copies of genes that code for Cas9 are introduced,
a protein or mRNA of maternally expressed endogenous Cas9 accumulates in the unfertilized egg cell during oogenesis,
the at least 3 copies of genes that code for Cas9 are expressed and genetically modify the nuclear genome, and
the protein or mRNA of maternally expressed endogenous Cas9 functions to the 2-cell stage from the fertilized egg.

2. A method for modifying at least one target gene, the method comprising:
introducing a guide RNA into at least one cell obtained from an unfertilized egg cell;
cleaving the at least one target gene at a cleavage site located upstream of a PAM sequence by Cas9 expressed in the at least one cell obtained from the unfertilized egg cell; and
obtaining at least one modified target gene in a region determined by complementary binding of the guide RNA and the at least one target gene,
wherein the at least one target gene has the PAM sequence,
the guide RNA comprises a polynucleotide comprising a base sequence complementary to an upstream base sequence of the PAM sequence in the at least one target gene,
the unfertilized egg cell is obtained from a genetically modified non-human organism comprising a nuclear genome into which at least 3 copies of genes that code for Cas9 are introduced,
a protein or mRNA of maternally expressed endogenous Cas9 accumulates in the unfertilized egg cell during oogenesis,
the at least 3 copies of genes that code for Cas9 are expressed and genetically modify the nuclear genome, and
the protein or mRNA of maternally expressed endogenous Cas9 functions to the 2-cell stage from the fertilized egg.

3. A method for modifying at least one target gene, the method comprising:
introducing a guide RNA into at least one cell obtained from an unfertilized egg cell;
cleaving the at least one target gene at a cleavage site located three bases upstream of a PAM sequence by Cas9 expressed in the at least one cell obtained from the unfertilized egg cell; and
obtaining at least one modified target gene in a region determined by complementary binding of the guide RNA and the at least one target gene,
wherein the at least one target gene has the PAM sequence comprising NGG of wherein N is any one base selected from the group consisting of adenine, cytosine, thymine, and guanine,
the guide RNA comprises a polynucleotide comprising base sequences complementary to base sequences from one base upstream to 20 bases to 24 bases upstream of the PAM sequence in the at least one target gene,
the unfertilized egg cell is obtained from a genetically modified non-human organism comprising a nuclear genome into which at least 3 copies of genes that code for Cas9 are introduced,
a protein or mRNA of maternally expressed endogenous Cas9 accumulates in the unfertilized egg cell during oogenesis,
the at least 3 copies of genes that code for Cas9 are expressed and genetically modify the nuclear genome, and
the protein or mRNA of maternally expressed endogenous Cas9 functions to the 2-cell stage from the fertilized egg.

4. The method of claim 1,
to wherein the introducing comprises introducing two or more of the guide RNAs into the at least one cell.

5. The method of claim 1,
wherein the introducing comprises introducing a foreign gene along with the guide RNAs into the at least one cell.

6. The method of claim 5,
wherein the foreign gene does not have the PAM sequence.

7. The method of claim 1,
wherein the unfertilized egg cell does not have the nuclear genome into which the Cas9 genes are introduced, and
the protein or mRNA of maternally expressed endogenous Cas9 accumulates in an oocyte during oogenesis.

8. A method for producing an unfertilized egg cell, the method comprising:
harvesting an unfertilized egg cell from a genetically modified non-human organism comprising a nuclear genome into which at least 3 copies of genes that code for Cas9 are introduced,
wherein a protein or mRNA of maternally expressed endogenous Cas9 accumulates in the unfertilized egg cell during oogenesis,
the at least 3 copies of genes that code for Cas9 are expressed and genetically modify the nuclear genome, and
the protein or mRNA of maternally expressed endogenous Cas9 functions to the 2-cell stage from the fertilized.

9. The method of claim 8,
wherein the unfertilized egg cell does not have the nuclear genome into which the Cas9 genes are introduced, and
the protein or mRNA of maternally expressed endogenous Cas9 accumulates in an oocyte during oogenesis.


SEQUENCE LISTING (82 sequences)

SEQ ID NO: 1 - protein, 7 aa, Artificial Sequence
  Synthesized
PKKKRKV

SEQ ID NO: 2 - protein, 16 aa, Artificial Sequence
  Synthesized
KRPAATKKAGQAKKKK

SEQ ID NO: 3 - protein, 9 aa, Artificial Sequence
  Synthesized
PAAKRVKLD

SEQ ID NO: 4 - protein, 11 aa, Artificial Sequence
  Synthesized
RQRRNELKRSP

SEQ ID NO: 5 - protein, 38 aa, Artificial Sequence
  Synthesized
NQSSNFGPMKGGNFGGRSSGPYGGGGQYFAKPRNQGGY

SEQ ID NO: 6 - protein, 42 aa, Artificial Sequence
  Synthesized
RMRIZFKNKGKDTAELRRRRVEVSVELRKAKKDEQILKRRNV

SEQ ID NO: 7 - protein, 8 aa, Artificial Sequence
  Synthesized
VSRKRPRP

SEQ ID NO: 8 - protein, 8 aa, Artificial Sequence
  Synthesized
PPKKARED

SEQ ID NO: 9 - protein, 8 aa, Artificial Sequence
  Synthesized
PQPKKKPL

SEQ ID NO: 10 - protein, 12 aa, Artificial Sequence
  Synthesized
SALIKKKKKMAP

SEQ ID NO: 11 - protein, 5 aa, Artificial Sequence
  Synthesized
DRLRR

SEQ ID NO: 12 - protein, 6 aa, Artificial Sequence
  Synthesized
PKQKKR

SEQ ID NO: 13 - protein, 10 aa, Artificial Sequence
  Synthesized
RKLKKKIKKL

SEQ ID NO: 14 - protein, 10 aa, Artificial Sequence
  Synthesized
REKKKFLKRR

SEQ ID NO: 15 - protein, 20 aa, Artificial Sequence
  Synthesized
KRKGDEVDGVDEVAKKKSKK

SEQ ID NO: 16 - protein, 17 aa, Artificial Sequence
  Synthesized
RKCLQAGMNLEARKTKK

SEQ ID NO: 17 - DNA, 4299 bp, Artificial Sequence
  Synthesized
atgggacctaagaaaaagaggaaggtggccgccgctgactacaaggatgacgacgataaatctagactggaacccggcgagaagccatacaaatgcccagagtgtggtaaatctttctctcagtctggtgctctgaccagacaccagcggacccacactagagacaagaaatactctattggactggatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtga

SEQ ID NO: 18 - DNA, 23 bp, Artificial Sequence
  Synthesized
cgcagatgacagggtaaggaagg

SEQ ID NO: 19 - DNA, 23 bp, Artificial Sequence
  Synthesized
atatgtattcgatcccacgttgg

SEQ ID NO: 20 - DNA, 23 bp, Artificial Sequence
  Synthesized
atgtgatccgttggcaacttagg

SEQ ID NO: 21 - DNA, 23 bp, Artificial Sequence
  Synthesized
gcgcgtcgtaccgtatggactgg

SEQ ID NO: 22 - DNA, 23 bp, Artificial Sequence
  Synthesized
gagaaaataatgaatgtcaaagg

SEQ ID NO: 23 - DNA, 23 bp, Artificial Sequence
  Synthesized
gggactgcgggtcggcatgacgg

SEQ ID NO: 24 - DNA, 23 bp, Artificial Sequence
  Synthesized
ggctgccagacaacgtgccgggg

SEQ ID NO: 25 - DNA, 23 bp, Artificial Sequence
  Synthesized
ggtcagcacccgcgtgggcatgg

SEQ ID NO: 26 - DNA, 23 bp, Artificial Sequence
  Synthesized
gctctgcttgccatggccccggg

SEQ ID NO: 27 - DNA, 23 bp, Artificial Sequence
  Synthesized
ggtgagcaccgtgtcgggcccgg

SEQ ID NO: 28 - DNA, 20 bp, Artificial Sequence
  Synthesized
cggtgaagggtgttgtgtga

SEQ ID NO: 29 - DNA, 21 bp, Artificial Sequence
  Synthesized
acacagtcatcaagcacaagc

SEQ ID NO: 30 - DNA, 24 bp, Artificial Sequence
  Synthesized
ctaacgcctgctggtggtcacagt

SEQ ID NO: 31 - DNA, 20 bp, Artificial Sequence
  Synthesized
tgtctagcccctgcttctct

SEQ ID NO: 32 - DNA, 25 bp, Artificial Sequence
  Synthesized
gctctagagcctctgctaaccatgt

SEQ ID NO: 33 - DNA, 32 bp, Artificial Sequence
  Synthesized
gcggaactccatatatgggctatgaactaatg

SEQ ID NO: 34 - DNA, 21 bp, Artificial Sequence
  Synthesized
cgccgtgctgttcttttgagc

SEQ ID NO: 35 - DNA, 28 bp, Artificial Sequence
  Synthesized
gccaccatggacaagaagtactccattg

SEQ ID NO: 36 - DNA, 28 bp, Artificial Sequence
  Synthesized
ccatcgattcacaccttcctcttcttct

SEQ ID NO: 37 - DNA, 24 bp, Artificial Sequence
  Synthesized
aagcgaattctccaaaagagtgat

SEQ ID NO: 38 - DNA, 20 bp, Artificial Sequence
  Synthesized
agagtttgctgaggcaggtc

SEQ ID NO: 39 - DNA, 20 bp, Artificial Sequence
  Synthesized
ccagcccatgtctcttccta

SEQ ID NO: 40 - DNA, 20 bp, Artificial Sequence
  Synthesized
tggtgtggctgtagtccttc

SEQ ID NO: 41 - DNA, 20 bp, Artificial Sequence
  Synthesized
caagtcaaaggggcctccac

SEQ ID NO: 42 - DNA, 21 bp, Artificial Sequence
  Synthesized
tcaaccacacagctgtttctc

SEQ ID NO: 43 - DNA, 22 bp, Artificial Sequence
  Synthesized
ctggcaccagtcacagggaatg

SEQ ID NO: 44 - DNA, 21 bp, Artificial Sequence
  Synthesized
gttgttataatcttctaacag

SEQ ID NO: 45 - DNA, 21 bp, Artificial Sequence
  Synthesized
ctgggtggaacaattgatcgt

SEQ ID NO: 46 - DNA, 18 bp, Artificial Sequence
  Synthesized
tgagccattgctgaggcg

SEQ ID NO: 47 - DNA, 18 bp, Artificial Sequence
  Synthesized
cgcgcctgatccttcctg

SEQ ID NO: 48 - DNA, 25 bp, Artificial Sequence
  Synthesized
tgttggatacaggccagactttgtt

SEQ ID NO: 49 - DNA, 25 bp, Artificial Sequence
  Synthesized
tcttaggctttgtatttggcttttc

SEQ ID NO: 50 - DNA, 20 bp, Artificial Sequence
  Synthesized
acgcaggtaccaaccaatct

SEQ ID NO: 51 - DNA, 20 bp, Artificial Sequence
  Synthesized
cccgggttcttcccttatgt

SEQ ID NO: 52 - DNA, 20 bp, Artificial Sequence
  Synthesized
ggtacgcgctctcttaggtt

SEQ ID NO: 53 - DNA, 20 bp, Artificial Sequence
  Synthesized
gcgtgtttcagatcgtctcc

SEQ ID NO: 54 - DNA, 20 bp, Artificial Sequence
  Synthesized
gtcagagcgaggtgctgagt

SEQ ID NO: 55 - DNA, 23 bp, Artificial Sequence
  Synthesized
cccatcaccactctactgtcttg

SEQ ID NO: 56 - DNA, 19 bp, Artificial Sequence
  Synthesized
gcactggtggtctggagga

SEQ ID NO: 57 - DNA, 21 bp, Artificial Sequence
  Synthesized
ccctcatcacctgggatacct

SEQ ID NO: 58 - DNA, 19 bp, Artificial Sequence
  Synthesized
tgtgcctccctccgctgtt

SEQ ID NO: 59 - DNA, 21 bp, Artificial Sequence
  Synthesized
ttactcctccacaccacaaga

SEQ ID NO: 60 - DNA, 18 bp, Artificial Sequence
  Synthesized
ccgagctggaagcgagag

SEQ ID NO: 61 - DNA, 18 bp, Artificial Sequence
  Synthesized
gacctctccgtccggtct

SEQ ID NO: 62 - DNA, 29 bp, Artificial Sequence
  Synthesized
tcgaattcatcttagttggccatgaagac

SEQ ID NO: 63 - DNA, 31 bp, Artificial Sequence
  Synthesized
tcgaattcacagcagccgatcagtgtgcttg

SEQ ID NO: 64 - DNA, 25 bp, Artificial Sequence
  Synthesized
gtggctgaatgagactggtgtcgac

SEQ ID NO: 65 - DNA, 61 bp, Artificial Sequence
  Synthesized
cgaagagtaaccgttgctaggagagaccgtggctgaatgagactggtgtcgacactagtgg

SEQ ID NO: 66 - DNA, 44 bp, Artificial Sequence
  Synthesized
ccactagtgtcgacaccagtctctaattttttttttcaaaaaaa

SEQ ID NO: 67 - DNA, 25 bp, Artificial Sequence
  Synthesized
tatatctgttttccaaccccagctc

SEQ ID NO: 68 - DNA, 25 bp, Artificial Sequence
  Synthesized
tggggtacacgtagaccttttatga

SEQ ID NO: 69 - DNA, 26 bp, Artificial Sequence
  Synthesized
tctctggctgaattgttttacacagg

SEQ ID NO: 70 - DNA, 23 bp, Artificial Sequence
  Synthesized
aaagcagtcagcgaacctttttg

SEQ ID NO: 71 - DNA, 23 bp, Artificial Sequence
  Synthesized
atgtctcgccagtccagtgtgtc

SEQ ID NO: 72 - DNA, 23 bp, Artificial Sequence
  Synthesized
caccaccaaaaccaaatccactg

SEQ ID NO: 73 - DNA, 23 bp, Artificial Sequence
  Synthesized
actcagacgtcttcgcacctgtc

SEQ ID NO: 74 - DNA, 23 bp, Artificial Sequence
  Synthesized
cgccacatagtcggagttgtaga

SEQ ID NO: 75 - DNA, 23 bp, Artificial Sequence
  Synthesized
cacctctgcagatgagggaagtg

SEQ ID NO: 76 - DNA, 23 bp, Artificial Sequence
  Synthesized
gggcatcacctcgcagcttatag

SEQ ID NO: 77 - DNA, 22 bp, Artificial Sequence
  Synthesized
cgcctttgacatgaaccaagtg

SEQ ID NO: 78 - DNA, 23 bp, Artificial Sequence
  Synthesized
actcactgtagcctcgggacctc

SEQ ID NO: 79 - DNA, 44 bp, Artificial Sequence
  Synthesized
cccgtaatccccagaccgtccatgcccacgcgggtgctgaccat

SEQ ID NO: 80 - DNA, 1067 bp, Artificial Sequence
  Synthesized
tgtacaaactgcgccgcagccctgcgcgcgaagctcctggcccgcgaggggtgcggtcggcccgtcggaggcgggacctcgtgggccttctttcctactggctaccctctggccctggggccttgccattggccacaacgctgccagtccggtgagctggccctgcctccccggagttgggtgaaatagaggcgggcgtcaagtgtcagtagtcgcggggcaggtacgcgctctcttaggttgctggtggagcttgggggtggcgggagcgggttccggcgagagggtgagcgagcgctggggcggggcgggagaaagtggctgcccgaggacctcggcggtgacgcgtggccgagcccaggagccccgatctccgtgcccgccttcgtgagcgtctggctgccggcccaggggtcccccgccgcggccccccgccgagtccgccgtcccgtgccagcccgagcgaggtgggatcgcgatcgctccgtgtcccgctcccgtaatccccagaccggccatggactacaaagaccatgacggtgattataaagatcatgacatcgattacaaggatgacgatgacaagggaggatacccctacgacgtgcccgactacgccggaggacccacccgggtgctgaccatgagcgcccgcctgggaccactgccccagccgccggccgcgcaggacgagcccgtgttcgcgcagctcaagccggtgctgggcgctgcgaacccggcccgcgacgcggcgctcttctccggagacgatctgaaacacgcgcaccaccacccgcctgcgccgccgccagccgctggcccgcgactgccctcggaggagctggtccaggtaagcagagcggccggcctcccttccccggctcgggcgtgtcccgccgctacgaggcgcgggcgacaggggccgctccagactgggacgcgcgggaacgggtgcccgggtggacagccccgccctgcgctcccagacggggcgccggggagtggatgggggagcagggcgggggcccacccggatctgtccagaggagctaaagagaggcggccgc

SEQ ID NO: 81 - DNA, 30 bp, Artificial Sequence
  Synthesized
ctgcaggaattcgccaccatgggacctaag

SEQ ID NO: 82 - DNA, 31 bp, Artificial Sequence
  Synthesized
gctgtttgtgccgatagccagtccaatagag